(19) United States
(12) Patent Application Publication
(10) Pub. No.: US 2023/0274811 A1
(43) Pub. Date: Aug. 31, 2023

(54) METHODS OF MAKING PROSTATE CANCER TREATMENT DECISIONS

(71) Applicant: Progenics Pharmaceuticals, Inc., New York, NY (US)

(72) Inventor: Vivien Wong, Scarsdale, NY (US)

(73) Assignee: Progenics Pharmaceuticals, Inc., New York, NY (US)

(21) Appl. No.: 18/094,898

(22) Filed: Jan. 9, 2023

Related U.S. Application Data
(63) Continuation of application No. PCT/US21/41000, filed on Jul. 9, 2021.
(60) Provisional application No. 63/090,561, filed on Oct. 12, 2020.
(60) Provisional application No. 63/051,797, filed on Jul. 14, 2020.
(60) Provisional application No. 63/050,371, filed on Jul. 10, 2020.

(51) Int. Cl. G16H 20/40; G16H 20/17; G16H 50/20; G16H 10/20

(52) U.S. Cl. CPC G16H 20/40; G16H 20/17; G16H 50/20; G16H 10/20

(57) ABSTRACT

Provided herein are methods for assessing a subject with prostate cancer. The methods are also directed to or may further comprise treating the subject and/or making treatment management decisions based on the assessing.

### RELATED APPLICATIONS

This application claims the benefit of priority under 35 U.S.C. § 119(e) to U.S. Provisional Application Nos. 63/050,371, filed Jul. 10, 2020; 63/051,797, filed Jul. 14, 2020; and 63/090,561, filed Oct. 12, 2020; the entire contents of each which are incorporated herein by reference.

### BACKGROUND OF THE INVENTION

Prostate cancer is the second most common form of cancer affecting men in the United States: an estimated one in seven men will be diagnosed with prostate cancer in his lifetime. The American Cancer Society estimates that each year approximately 164,609 new cases of prostate cancer will be diagnosed, and about 26,730 men will die of the disease. Approximately 2.9 million men in the U.S. currently count themselves among prostate cancer survivors. New methods are needed to expand treatment and treatment management options.

### SUMMARY OF THE INVENTION

In one aspect, a method of treating, or making a treatment management decision for, prostate cancer in a subject, the method comprising imaging the subject with ¹⁸F-DCFPyL PET/CT is provided. In one embodiment of such methods, the imaging is for determining the presence or absence of one or more prostate cancer lesions, such as in some embodiments, metastases in the subject with ¹⁸F-DCFPyL PET/CT is provided. In one embodiment, the prostate cancer lesions occur in the prostate. In one embodiment of any one of the methods provided herein, the imaging is for determining the stage of prostate cancer, such as in subjects with high risk prostate cancer, for nodal or distant metastases being evaluated for initial therapy, or in subjects with prostate cancer recurrence or metastases following course(s) of therapy, in the subject with ¹⁸F-DCFPyL PET/CT is provided. In one embodiment of such methods, the method further comprises treating or making a treatment management decision for the subject based on the imaging, determining, presence or absence of one or more prostate cancer lesions, such as one or more metastases, and/or stage of the prostate cancer. In one embodiment of any one of the methods provided herein, the determining the presence or absence of one or more metastases or the stage of prostate cancer is based on obtaining or being given the results of any one of the methods of imaging or assessment provided herein. In one embodiment of any one of the methods provided herein, the determining the presence or absence of one or more metastases or the stage of prostate cancer is based on performing any one of the methods of imaging or assessment provided herein.

In one embodiment of any one of the methods provided herein, the imaging is for determining the presence or absence of one or more prostate cancer lesions, such as one or more metastases, and the treating or making a treatment management decision is based on the presence or absence of the one or more prostate cancer lesions, such as one or more metastases.

In one embodiment of any one of the methods provided herein, the imaging is for determining the stage of the prostate cancer in the subject. In one embodiment of any one of the methods provided herein, the subject has stage T, N (e.g., N1) or M1 (e.g., M1a, M1b, M1c) prostate cancer.

In one embodiment of any one of the methods provided herein, the subject is any one of the subjects provided herein, such as one with metastatic or recurrent prostate cancer, or is suspected of having metastatic or recurrent prostate cancer. In one embodiment of any one of the methods provided herein, the subject has biochemically recurrent prostate cancer. In one embodiment of any one of the methods provided herein, the subject has high risk prostate cancer.

As used herein, “high risk prostate cancer” is as defined by NCCN Guidelines Version 3.2016 (clinical stage ≥T3a or PSA >20 ng/mL or Gleason score ≥8). A high risk prostate cancer subject can also have biopsy confirmed presence of adenocarcinoma of the prostate gland and/or be scheduled or will be scheduled to undergo radical prostatectomy with pelvic lymph node dissection.

In one embodiment of any one of the methods provide herein, any one of the subjects may have a PSA level of less than 2, less than 1.5, less the 1 or less than 0.5 ng/mL. In one embodiment of any one of the methods provide herein, any one of the subjects may have a PSA level of between 0.2-0.5 ng/mL, 0.5-1 ng/mL, 0.5-2 ng/ml, 1-2 ng/mL or greater than or equal to 2 ng/mL. In one embodiment of any one of the methods provide herein, any one of the subjects may have a PSA level of between 2-5 or greater than or equal to 5 ng/mL. In one embodiment of any one of the methods provide herein, any one of the subjects may have a PSA level of greater than or equal to 0.2 ng/mL or is between 0.2-5 ng/mL. In an embodiment of any one of the foregoing the PSA level can be less than or equal to 5 ng/mL. In one embodiment of any one of the methods provide herein, any one of the subjects may have a rising PSA level or a change in PSA levels after a prostate cancer therapy and/or negative or equivocal imaging.

In one embodiment of any one of the methods provided herein, the subject has nonmetastatic relapsed prostate cancer, or is suspected of having nonmetastatic relapsed prostate cancer.

In one embodiment of any one of the methods provided herein, the subject is one with prostatic lesions, or is suspected of having prostatic lesions. In one embodiment of any one of the methods provided herein, the subject is one with prostate bed lesions, or is suspected of having prostate bed lesions. In one embodiment of any one of the methods provided herein, the subject is one with extra-prostatic metastases, or is suspected of having extra-prostatic metastases. In one embodiment of any one of the methods provided herein, the subject is one with pelvic (e.g., lymph node) metastases, or is suspected of having pelvic (e.g., lymph node) metastases. In one embodiment of any one of the methods provided herein, the subject is one with extra-pelvic (e.g., lymph node) metastases, or is suspected of having extra-pelvic (e.g., lymph node) metastases. In one embodiment of any one of the methods provided herein, the subject is one with distant metastases, or is suspected of having distant metastases. In one embodiment of any one of the methods provided herein, this subject is one with, or is suspected of having nodal, bone, visceral organ, soft tissue, iliac, retroperitoneal para/peri aortic lymph node, limited osseous, rectal or peri-rectal, thoracic spine or lumbar metastases. In one embodiment of any one of the methods provided herein, the subject is one with nodal metastases.

In one embodiment of any one of the methods provided herein, the subject is one that meets any one of the set of inclusion and/or exclusion criteria provided herein.

In one embodiment of any one of the methods provided herein, the one or more of the metastases is greater than 4 mm. In one embodiment of any one of the methods provided herein, the median lymph node metastases are >4 mm.

In one embodiment of any one of the methods provided herein, ¹⁸F-DCFPyL is administered 1-2 hours prior to PET/CT to the subject.

In one embodiment of any one of the methods provided herein, 9 mCi (333 MBq) of ¹⁸F-DCFPyL is administered by IV injection to the subject. In one embodiment of any one of the methods provided herein, 9 mCi (333 MBq) of ¹⁸F-DCFPyL is administered by a single IV bolus injection in a total volume of 10 mL or less to the subject.

In one embodiment of any one of the methods provided herein, the method further comprises treating the subject based on the imaging, determining the presence or absence of one or more metastases, determining the stage of prostate cancer, etc. In one embodiment of any one of the methods provided herein, the treatment is any one of the treatments provided herein.

In one embodiment of any one of the methods provided herein, the method further comprises making a treatment management decision. In one embodiment of any one of the methods provided herein, the making a treatment management decision includes any one or more of the treatment management decisions provided herein.

In one embodiment of any one of the methods provided herein, the making a treatment management decision comprises the decision to use radiation, focal, salvage or systemic therapy. In one embodiment of any one of the methods provided herein, the making a treatment management decision comprises the decision to observe the subject. In one embodiment of any one of the methods provided herein, the making a treatment management decision comprises the decision to change the treatment of the subject. In one embodiment of any one of the methods provided herein, the change is from systemic to salvage local therapy. In one embodiment of any one of the methods provided herein, the change is from salvage local therapy to systemic therapy. In one embodiment of any one of the methods provided herein, the change is from observation to a treatment. In one embodiment of any one of the methods provided herein, the change is from a treatment to observation. In one embodiment of any one of the methods provided herein, the change is from radiation to hormone/ADT therapy. In one embodiment of any one of the methods provided herein, the making a treatment management decision comprises the decision to forego a treatment of the subject. In one embodiment of any one of the methods provided herein, the decision is to forego hormone/ADT therapy.

In one embodiment of any one of the methods provided herein, the subject has undergone a prior diagnostic test, such as a PSA test or a conventional imaging test. In one embodiment of any one of the methods provided herein, the prior diagnostic test was negative or equivocal findings for prostate cancer on conventional imaging.

In one embodiment of any one of the methods provided herein, the method further comprises a step of performing an additional diagnostic test on the subject, such as a PSA test or conventional imaging test. In one embodiment of any one of the methods provided herein, the PSA test result was detectable or rising PSA post-radical prostatectomy in the subject. In one embodiment of any one of the methods provided herein, the PSA test result was an increase in PSA level post-radiation therapy, cryotherapy, or brachytherapy in the subject.

In one embodiment of any one of the methods provided herein, the method further comprises completing pre- and/or post-imaging a questionnaire, such as of Example 10, respectively.

In one embodiment of any one of the methods provided herein, the diagnostic performance of the ¹⁸F-DCFPyL PET/CT imaging, such as when centrally read, compared to centrally read conventional imaging (e.g., for determining the presence or absence of one or more prostate cancer lesions, such as one or more metastases, or determining the stage of the prostate cancer in the subject) has similar or increased PPV, NPV, specificity, and/or sensitivity.

In one embodiment of any one of the methods provided herein, the PPV is at least 2-fold or 3-fold higher. In one embodiment of any one of the methods provided herein, the specificity is greater than 90% or 95%.

In one embodiment of any one of the methods provided herein, the diagnostic performance of the ¹⁸F-DCFPyL PET/CT imaging, such as when centrally read, compared to centrally read conventional imaging for determining pelvic lymph node metastases (N1) demonstrates at least 2-fold or 3-fold higher PPV, improved NPV, high or near-perfect specificity (e.g., at least 95%, 96% or 97%, or 97.9% vs. 67.3%, respectively), and/or similar or greater sensitivity (e.g., ˜40% or at least 40%).

In one embodiment of any one of the methods provided herein, the diagnostic performance of the ¹⁸F-DCFPyL PET/CT imaging, such as when centrally read, compared to centrally read conventional imaging for determining pelvic lymph node (N1) demonstrates a PPV greater than 2-fold higher, improved NPV, and a specificity greater than 90% or 95%.

In one aspect, a composition of ¹⁸F-DCFPyL of 1-90 mCi/mL (37-3330 MBq/mL) at End of Synthesis (EOS) is provided. In one embodiment of any one of the compositions provided herein, the composition is a clear, particulate-free injectable solution, such as at a strength as provided herein. In one embodiment of any one of the compositions provided herein, the composition is in an unit-dose syringe.

In any one of the methods provided herein, the PPV is any one of the PPV values provided herein.

In any one of the methods provided herein, the NPV is any one of the NPV values provided herein.

In any one of the methods provided herein, the sensitivity value is any one of the sensitivity values provided herein.

In any one of the methods provided herein, the specificity value is any one of the specificity values provided herein.

In any one of the methods provided herein, the increase (as compared to conventional imaging) in the PPV is equal to or at least any one of the increases in PPV values provided herein.

In any one of the methods provided herein, the increase (as compared to conventional imaging) in the NPV is equal to or at least any one of the increases in NPV values provided herein.

In any one of the methods provided herein, the increase (as compared to conventional imaging) in the sensitivity value is equal to or at least any one of the increases in the sensitivity values provided herein.

In any one of the methods provided herein, the increase (as compared to conventional imaging) in the specificity value is equal to or at least any one of the increases in the specificity values provided herein.

In one aspect, a method of using ¹⁸F-DCFPyL to image men with prostate cancer, comprising (i) contacting a subject, such as any one of the subjects provided herein, with ¹⁸F-DCFPyL or PyLARIFY, and (ii) imaging the subject, such as with PET/CT imaging, wherein a study of diagnostic performance of the ¹⁸F-DCFPyL PET/CT imaging, such as when centrally read, compared to centrally read conventional imaging (e.g., for determining the presence or absence of one or more prostate cancer lesions, such as one or more metastases, or determining the stage of the prostate cancer in the subject) has similar or increased PPV, NPV, specificity, and/or sensitivity, is provided.

In any one of the methods provided herein, the PPV is at least 2-fold or 3-fold higher. In any one of the methods provided herein, the specificity is greater than 90% or 95%.

In any one of the methods provided herein, the study of the diagnostic performance of the ¹⁸F-DCFPyL PET/CT imaging, such as when centrally read, compared to centrally read conventional imaging for determining pelvic lymph node metastases (N1) demonstrates at least 2-fold or 3-fold higher PPV, improved NPV, high or near-perfect specificity (e.g., at least 95%, 96% or 97%, or 97.9% vs. 67.3%, respectively), and/or similar or greater sensitivity (e.g., ˜40% or at least 40%).

In any one of the methods provided herein, the study of the diagnostic performance of the ¹⁸F-DCFPyL PET/CT imaging, such as when centrally read, compared to centrally read conventional imaging for determining pelvic lymph node (N1) demonstrates a PPV greater than 2-fold higher, improved NPV, and a specificity greater than 90% or 95%.

In one aspect, a method of restaging prostate cancer in a subject, said prostate cancer initially staged with conventional imaging, the method comprising: imaging the subject with ¹⁸F-DCFPyL PET/CT, and restaging the prostate cancer in the subject based on the imaging with ¹⁸F-DCFPyL PET/CT, wherein the subject is one with metastatic or recurrent prostate cancer, or is suspected of having metastatic or recurrent prostate cancer; or has biochemically recurrent prostate cancer, or is suspected of having biochemically recurrent prostate cancer; or has nonmetastatic relapsed prostate cancer, or is suspected of having nonmetastatic relapsed prostate cancer, is provided.

In any one of the methods provided herein, the method further comprises comparing the restage determined by the imaging with ¹⁸F-DCFPyL PET/CT with the stage initially determined with conventional imaging.

In any one of the methods provided herein, the subject is up-staged (e.g., from M0 to M1, M1a to M1b or M1c, or M1b to M1c) based on the comparing. In any one of the methods provided herein, the subject is down-staged (e.g., from M1 to M0) based on the comparing.

In any one of the methods provided herein, the method further comprises treating or making a treatment management decision for the subject based on the restage determined by the imaging with ¹⁸F-DCFPyL PET/CT and/or the comparing.

In any one of the methods provided herein, the making a treatment management decision includes any one or more of the treatment management decisions provided herein. In any one of the methods provided herein, the making a treatment management decision comprises the decision to use radiation, focal, salvage or systemic therapy. In any one of the methods provided herein, the making a treatment management decision comprises the decision to observe the subject. In any one of the methods provided herein, the making a treatment management decision comprises the decision to change the treatment of the subject. In any one of the methods provided herein, the change is from systemic to salvage local therapy. In any one of the methods provided herein, the change is from salvage local therapy to systemic therapy. In any one of the methods provided herein, the change is from observation to a treatment. In any one of the methods provided herein, the change is from a treatment to observation. In any one of the methods provided herein, the change is from radiation to hormone/ADT therapy. In any one of the methods provided herein, the making a treatment management decision comprises the decision to forego a treatment of the subject. In any one of the methods provided herein, the decision is to forego hormone/ADT therapy.

In any one of the methods provided herein, ¹⁸F-DCFPyL is administered 1-2 hours prior to PET/CT to the subject. In any one of the methods provided herein, 9 mCi (333 MBq) of ¹⁸F-DCFPyL is administered by a single IV bolus injection in a total volume of 10 mL or less to the subject.

In any one of the methods provided herein, the method further comprises treating the subject based on the restage determined by the imaging with ¹⁸F-DCFPyL PET/CT and/or the comparing.

In any one of the methods provided herein, the treatment is any one of the treatments provided herein.

In any one of the methods provided herein, the subject is any one of the subjects as described herein.

In any one of the methods provided herein, the subject has a PSA level of less than 2 ng/ml.

In any one of the methods provided herein, the method further comprises completing pre- and/or post-imaging a questionnaire, such as of Example 10, respectively.

In one aspect, ¹⁸F-DCFPyL for use in a method of treating, or making a treatment management decision for, prostate cancer in a subject, the method comprising: imaging the subject with ¹⁸F-DCFPyL PET/CT, and treating or making a treatment management decision for the subject based on the imaging, wherein the subject is one with metastatic or recurrent prostate cancer, or is suspected of having metastatic or recurrent prostate cancer; or has biochemically recurrent prostate cancer, or is suspected of having biochemically recurrent prostate cancer; or has a PSA level of less than 2 ng/ml; or has nonmetastatic relapsed prostate cancer, or is suspected of having nonmetastatic relapsed prostate cancer, is provided.

In one embodiment, ¹⁸F-DCFPyL is for use wherein: (a) the imaging is as defined in any one embodiment herein; (b) the subject is as defined in any one embodiment herein; (c) the ¹⁸F-DCFPyL is administered as defined in any one embodiment herein; (d) the method is as defined in any one embodiment herein; (e) the subject is as defined in any one embodiment herein; (f) the method further comprises an additional diagnostic test as defined in any one embodiment herein; and/or (g) the diagnostic performance is as defined in any one embodiment herein.

In one aspect, ¹⁸F-DCFPyL for use in a method of imaging men with prostate cancer, comprising: (i) contacting a subject, such as any one of the subjects provided herein, with ¹⁸F-DCFPyL, and (ii) imaging the subject, such as with PET/CT imaging, is provided.

In one embodiment, ¹⁸F-DCFPyL is for use, wherein a study of diagnostic performance of the ¹⁸F-DCFPyL PET/CT imaging, such as when centrally read, compared to centrally read conventional imaging (e.g., for determining the presence or absence of one or more prostate cancer lesions, such as one or more metastases, or determining the stage of the prostate cancer in the subject) has similar or increased PPV, NP, specificity, and/or sensitivity.

In one aspect ¹⁸F-DCFPyL is for use according to any one embodiment herein, wherein the method is as defined in any one embodiment herein.

In one aspect, ¹⁸F-DCFPyL for use in a method of restaging prostate cancer in a subject, said prostate cancer initially staged with conventional imaging, the method comprising: imaging the subject with ¹⁸F-DCFPyL PET/CT, and restaging the prostate cancer in the subject based on the imaging with ¹⁸F-DCFPyL PET/CT, wherein the subject is one with metastatic or recurrent prostate cancer, or is suspected of having metastatic or recurrent prostate cancer; or has biochemically recurrent prostate cancer, or is suspected of having biochemically recurrent prostate cancer; or has nonmetastatic relapsed prostate cancer, or is suspected of having nonmetastatic relapsed prostate cancer, is provided.

In one embodiment, ¹⁸F-DCFPyL is for use, wherein the method is as defined in any one embodiment herein.

In one embodiment, ¹⁸F-DCFPyL is for use according to any one embodiment herein, wherein the ¹⁸F-DCFPyL is in the form of a sterile, clear, colourless solution for intravenous injection having a pH of 4.5 to 7.5, each milliliter of which contains: <1 microgram of DCFPyL, 37 to 2960 MBq (1 to 80 mCi) florcarpiroic F 18 at calibration time and date, and ≤78.9 mg ethanol in 0.9% sodium chloride injection USP.

In one aspect, use of ¹⁸F-DCFPyL for the manufacture of a medicament for use in a method as defined in any one embodiment provided herein, is provided.

### BRIEF DESCRIPTION OF THE DRAWINGS

FIG. 1 shows the OSPREY study design and cohorts.

FIG. 2 shows the diagnostic performance in pelvic lymph nodes.

FIG. 3 shows the study design. There were 14 sites in the US and CA. Central imaging core lab: three blinded, independent ¹⁸F-DCFPyL PET/CT readers; two separate truth panel readers, consensus read.

FIG. 4 shows select baseline characteristics.

FIG. 5 shows correct localization rate (CLR).

FIG. 6 shows CLR by baseline imaging modality.

FIG. 7 shows CLR by standard of truth (SOT).

FIG. 8 shows reader agreement.

FIG. 9 shows CLR (%) by PSA groups.

FIGS. 10A-10C show case study 1 results. FIG. 10A demonstrates case study 1: 71 yo man with Gleason 4+3=7 prostate cancer, post-RP, and EBRT, presenting with PSA 1.92 ng/mL and no evidence of disease on baseline ¹⁸F-FACBC PET/CT; ¹⁸F-D CFPyL PET/CT showed L common iliac and multiple retroperitoneal lymph node lesions; CT-guided biopsy of retroperitoneal LN was confirmed positively by pathology; No AEs were reported; and Intended management plan changed from observation (no therapy) to initiating ADT. FIGS. 10B-10C show scan results.

FIGS. 11A-11C show case study 2 results. FIG. 11A demonstrates case study 1: 76 yo man with Gleason 4+5=9 prostate cancer, post-RP with PLND and ADT, presenting with PSA 1.27 ng/mL and no evidence of disease on baseline ¹⁸F-FACBC PET/CT; ¹⁸F-D CFPyL PET/CT showed lumbar spine metastasis which was confirmed by targeted contrast-MRI of the lumbar spine; No AEs were reported; and Intended management plan changed from local RT only to ADT+abiraterone+modification of the planned RT portals. FIGS. 11B-11C show scan results.

FIGS. 12A-12F show case study 3 results. FIG. 12A demonstrates case study 1: 69 yo man with Gleason 4+4=8 prostate cancer, history of radical prostatectomy with pelvic lymph node dissection, now with PSA of 1.78 and no evidence of disease by baseline conventional imaging with ¹¹C-Choline PET and MRI of the pelvis with contrast; ¹⁸F-D CFPyL scan showed a thoracic spine lesion which was confirmed by biopsy to be positive for prostate cancer; No AEs were reported; and there was a change in intended management of this patient with a change in the volume and dose of radiation therapy to be administered. FIGS. 12B-12F show scan results.

FIG. 13 shows results for Change of Intended Management.

FIG. 14 outlines safety. Similar safety profile to prior OSPREY study; and hypersensitivity was the single drug-related Grade 3 AE in a patient with significant allergic history.

FIG. 15 outlines the summary of case studies 1-3.

FIG. 16 shows STARD flow diagram with Composite Standard of Truth (SOT) validation.

FIG. 17 shows CLR by baseline PSA levels.

FIG. 18 shows detection rates by baseline PSA groups.

FIGS. 19A-19B show PPV by anatomic region (FIG. 19A) and extra-pelvic region (FIG. 19B).

FIG. 20 shows Change in Planned Medical Management.

FIG. 21 shows negative or equivocal baseline imaging.

FIG. 22 shows participant flow.

FIGS. 23A-23B show pre- and post-PyL medical management questionnaires respectively.

FIG. 24 shows the ¹⁸F-DCFPyL clinical development program.

FIGS. 25A-25C show results from a case study.

### DETAILED DESCRIPTION OF THE INVENTION

¹⁸F-DCFPyL, also referred to herein PyL, is a fluorinated PSMA-targeted Positron Emission Topography/Computed Tomography (“PET/CT”) imaging agent that enables visualization of prostate cancer lesions, such as in the prostate gland, pelvic lymph nodes, bone and soft tissue metastases. It has been surprisingly found that imaging with such an agent can be used to image prostate cancer patients with a high level of diagnostic performance, such as sensitivity, specificity, negative predictive value and/or positive predictive value of subjects as provided herein.

### Chemical Characteristics

¹⁸F-DCFPyL contains fluorine 18 (F 18), and is a radiolabeled prostate-specific membrane antigen inhibitor. Chemically, ¹⁸F-DCFPyL (USAN name:florcarpiroic F 18) is 2-(3-{1-carboxy-5-[(6-[¹⁸F]fluoro-pyridine-3-carbonyl)-amino]-pentyl}ureido)-pentanedioic acid. The molecular weight is generally 441.4, and the structural formula is:

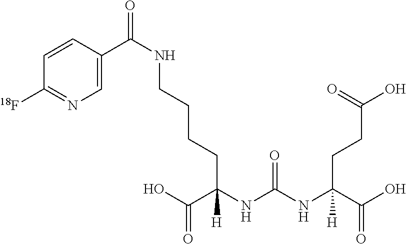 [SMILES: O=C(O)CC[C@H](NC(=O)N[C@@H](CCCCNC(=O)c1ccc([18F])nc1)C(=O)O)C(=O)O]

¹⁸F-DCFPyL can be provided as PyLARIFY®, which is a sterile, clear, colorless solution for intravenous injection. Each milliliter can contain, <1 microgram of DCFPyL, 37 to 2960 MBq (1 to 80 mCi) florcarpiroic F 18 at calibration time and date, and ≤78.9 mg ethanol in 0.9% sodium chloride injection USP. The pH of the solution is 4.5 to 7.5.

In one embodiment of any one of the methods provided herein, the ¹⁸F-DCFPyL, or the administration or imaging as a result thereof, has any one or more or all, or any combination, of the following:
(i) provided as a clear, colorless solution;
(ii) containing 37 MBq/mL to 2960 MBq/mL (1 mCi/mL to 80 mCi/mL) of florcarpiroic F 18 at end of synthesis;
(iii) amount of radioactivity administered for PET imaging of 333 MBq (9 mCi);
(iv) administered as a single bolus intravenous injection, such as in a total volume of 10 mL or less;
(v) start time for image acquisition is about 60 minutes (or 60-120 minutes) after injection;
(vi) image acquisition starting from mid-thigh to the skull vertex;
(vii) scan duration of 12 to 40 minutes; and
(viii) acquisition time per bed position of 2-5 minutes.

¹⁸F-DCFPyL binds to the active site of prostate specific membrane antigen (PSMA) which is overexpressed in prostate cancer cells. ¹⁸F-DCFPyL dose-dependently inhibits PSMA enzymatic activity in vitro, for example, with a Ki of 1.1±0.1 nM. In mouse xenografts, ¹⁸F-DCFPyL is taken up only in PSMA-positive PC3 PIP tumor and not in PSMA-negative PC3 flu tumors, demonstrating that the uptake of ¹⁸F-DCFPyL is PSMA specific.

A subject provided herein is one with, or is suspected of having, metastatic or recurrent prostate cancer on which PSMA is expressed. PSMA is expressed in increased amounts in prostate cancer (Rochon et al., 1994, Prostate 25:219-223; Murphy et al., 1995, Prostate 26:164-168; and Murphy et al., 1995, Anticancer Res. 15:1473-1479).

“Metastatic”, as used herein, refers to cancer, that has spread from the primary cancer or primary cancer site. In prostate cancer, the metastases is extra-prostatic. The metastases can occur within the pelvis, such as in the pelvic lymph nodes, or can be in a site distant from the pelvis (also referred to herein “distant metastases”). In one embodiment, of any one of the methods provided herein, the metastases are pelvic lymph node metastases or metastases in other distant sites. Thus, a metastases can be pelvic, nodal, extra-prostatic, extra-pelvic, in bone, or in visceral organs or soft tissue, etc.

“Recurrent”, as used herein, refers to prostate cancer that occurs after an initial prostate cancer therapy has occurred or refers to prostate cancer cells that have survived a treatment. “Biochemical recurrence refers to disease recurrence (rising PSA) after initial definitive therapy, defined as a confirmed PSA value of ≥0.2 ng/mL after prostatectomy or ≥2 ng/mL above nadir after radiation therapy. Generally, recurrent prostate cancer is one where there are one or more prostate cancer lesions in the subject. The recurrent prostate cancer lesions can be local, regional or distant. The prostate cancer lesions can be metastases.

“Nonmetastatic relapsed” prostate cancer refers to recurrent prostate cancer that has not spread to other parts of the body and is no longer responding to a medical or surgical treatment, such as one that lowers testosterone. In other words, the prostate cancer can be considered castrate resistant.

“Staging”, as used herein refers to determining the extent and/or localization of the prostate cancer in the subject. The staging can determine the extent of nodal or metastatic disease. Thus, any one of the methods provided herein can be used to stage the prostate cancer in any one of the subjects provided herein. The method can be used to determine if the stage of the prostate cancer in the subject is, for example, prostatic (T), pelvic LN (N) (e.g., N1), extra-pelvic LN (M1a), bone (M1b) & other visceral organs/soft tissue (M1c). “Restaging” is any staging performed, such as with imaging with ¹⁸F-DCFPyL PET/CT, that occurs after prior, first or initial staging using some other method, such as conventional imaging. Restaging can include any staging performed after prostate cancer recurrence or metastases following course(s) of therapy. As used herein, “up-staging” refers to assigning a stage of prostate cancer, such as from imaging with ¹⁸F-DCFPyL PET/CT, to a subject that is more advanced or is associated with higher risk of a negative outcome than a stage prior, first or initially assigned to or determined with another method, such as with conventional imaging, in the subject. “Down-staging” refers to assigning a stage of prostate cancer, such as from imaging with ¹⁸F-DCFPyL PET/CT, to a subject that is less advanced or is associated with lower risk of a negative outcome than a stage prior, first or initially assigned to or determined with another method, such as with conventional imaging, in the subject.

In one aspect, a method of treating or making a treatment management decision for any one of the subjects provided herein comprising imaging the subject with ¹⁸F-DCFPyL PET/CT and treating or making a treatment management decision for the subject based on the imaging is provided. The method can be for determining the presence or absence of prostate cancer lesions, such as metastases, staging the prostate cancer, restaging the prostate cancer, etc.

“Treating, as used herein, refers to any clinical action taken or recommended to be taken to reduce or eliminate prostate cancer in a subject or to provide any benefit to the subject such as the reduction of symptoms as a result of having prostate cancer or to limit, inhibit or stop progression of prostate cancer in a subject. Treatment of prostate cancer includes, but is not limited to, focal therapy, systemic therapy, salvage therapy, surgery (e.g., prostatectomy), radiation, cryotherapy, brachytherapy, chemotherapy, or hormonal/androgen deprivation therapy (ADT) therapy. In any one of the methods provided herein, the treating or treatment of the subject can comprise any one of the forms of treatment provided herein or a recommendation to provide such a treatment to a subject. In any one of the methods provided herein, the treating or treatment of the subject can comprise a change in the volume and/or dose of any one of the therapies provided herein, such as radiation therapy. In any one of the methods provided herein, the treating or treatment of the subject can comprise ADT and abiraterone or ADT and abiraterone and a change in radiation therapy. In any one of the methods provided herein, the treating or treatment of the subject can comprise ADT and abiraterone or ADT and abiraterone and a change from radiation therapy.

In one embodiment of any one of the methods provided herein, the radiation is radiation of the prostate or prostate bed, pelvis or is extra-pelvic. In one embodiment of any one of the methods provided herein, the focal therapy is cryosurgery, high-intensity focused ultrasound or brachytherapy. In one embodiment of any one of the methods provided herein, the salvage therapy is prostatectomy, pelvic lymph node/lesion dissection or metastatectomy/distant lymph node/lesion dissection. In one embodiment of any one of the methods provided herein, the systemic therapy is androgen deprivation therapy, an antiandrogen drug (e.g., abiraterone, apalutamide, darolutamide, enzalutamide) or chemotherapy. In one embodiment of any one of the methods provided herein, treating or treatment of a subject comprises I-131 1095 in combination with enzalutamide.

In one embodiment of any one of the methods provided herein the salvage therapy is prostatectomy. In one embodiment of any one of the methods provided herein the treating or treatment management decision for T or N prostate cancer is pelvic lymph node/lesion dissection, metastatectomy/distant lymph node/lesion dissection and/or initiating a systemic therapy. In one embodiment of any one of the methods provided herein, the treating or treatment management decision for M disease is a systemic therapy.

“Making a treatment management decision”, as used herein, refers to any decision a clinician may make for a subject with prostate cancer in order to monitor and/or treat the prostate cancer in the subject. Treatment management decisions include, but are not limited to, determining that a biopsy be performed, changing the location to perform a biopsy, changing the frequency of performing a biopsy, determining that a surgery be performed, changing the type of surgery to be performed, changing the location to perform a surgery, changing the timing of performing a surgery, determining that radiation be administered, changing the type of radiation to administer, determining the dose of radiation to administer, determining the location (e.g., local, regional, locoregional) to which to administer radiation, changing the dose of radiation to administer, changing the location to which to administer radiation, determining that chemotherapy be performed, changing the type of chemotherapy to administer, determining the dose of chemotherapy to administer, changing the dose of chemotherapy to administer, changing the regimen for administering chemotherapy, determining hormonal/ADT therapy be administered, changing the type of hormonal/ADT therapy to administer, determining the dose of hormonal/ADT therapy to administer, changing the dose of hormonal/ADT therapy to administer, changing the regimen for administering hormonal/ADT therapy, and replacing a therapy or adding a supplemental therapy, such as replacing with systemic therapy or adding systemic therapy as a supplement to other therapy. In any one of the methods provided herein, the step of making a treatment management decision can include any one or more of the foregoing. In any one of the methods provided herein, the step of making a treatment management decision can include changing from a systemic therapy to a local or salvage local therapy, from a local or a salvage local therapy to a systemic therapy, from observation to initiating a therapy, from initiating or maintaining a therapy to observation. In any one of the methods provided herein, the method may further comprise a step of treating or monitoring the subject (or recommending the treatment or monitoring to the subject) according to the treatment management decision.

In any one of the methods provided herein, a step of making a treatment management decision can comprise completing a pre- and/or post-imaging with ¹⁸F-DCFPyL questionnaire, for example, one or both of the questionnaires provided herein, such as of Example 10.

The subject of any one of the methods provided herein can be one with metastatic or recurrent prostate cancer (or can be one in which metastatic or recurrent prostate cancer is suspected). The subject of any one of the methods provided herein can be one with biochemically recurrent prostate cancer (or can be one in which biochemical recurrence is suspected). In one embodiment of any one of the methods provided herein, the subject has high risk prostate cancer, such as according to the OSPREY clinical trial.

In some embodiments of any one of the methods provided, the subject has or may have a high or rising prostate-specific antigen (PSA) levels, such as one with detectable or rising PSA that is greater than or equal to 0.2 ng/mL with a confirmatory PSA of greater than or equal to 0.2 ng/mL. In some embodiments of any one of the methods provided, the subject may have a high or rising prostate-specific antigen (PSA) levels, such as one with elevated PSA that is greater than or equal to 0.2 ng/mL above the nadir. In some embodiments of any one of the methods provided, the subject has or may have a PSA level of less than 2, less than 1.5, less the 1 or less than 0.5 ng/mL. In some embodiments of any one of the methods provided, the subject has or may have a PSA level of between 0.2-0.5 ng/mL, 0.5-1 ng/mL, 0.5-2 ng/ml, 1-2 ng/mL or greater than or equal to 2 ng/mL. In some embodiments of any one of the methods provided, the subject has or may have a PSA level of between 2-5 or greater than or equal to 5 ng/mL. In some embodiments of any one of the methods provided, the subject has or may have a PSA level of greater than or equal to 0.2 ng/mL or is between 0.2-5 ng/mL. In an embodiment of any one of the foregoing the PSA level may also be less than or equal to 5 ng/mL. In one embodiment of any one of the methods provide herein, any one of the subjects may have a rising PSA level or a change in PSA levels after a prostate cancer therapy and/or negative or equivocal imaging.

In some embodiments of any one of the methods provided herein, the subject is one who has undergone PSA level testing or the method includes a step of testing the PSA level in the subject.

In some embodiments of such subjects, the subject may have undergone a radical prostatectomy. In some embodiments of any one of the methods provided herein, the subject is one who has undergone radical prostatectomy or the method includes a step of treating the subject with radical prostatectomy.

In some embodiments of any one of the methods provided, the subject may have negative or equivocal findings for prostate cancer with conventional imaging, such as conventional imaging performed as part of a standard of care workup. Conventional imaging methods include, but are not limited to ultrasound, CT/MRI (e.g., pelvic), PET/CT, computed tomography (CT), magnetic resonance imaging (MRI), bone scan (e.g., ^{99m}Tc nethylene diphosphonate (MDP) bone scan), whole-body scan, whole-body bone scan, whole-body bone scintigraphy, bone scintigraphy, NaF, fluciclovine (e.g., ¹⁸F-fluciclovine), ¹⁸F-FACBC (Axumin), ¹⁸F-FDG or choline (e.g., ¹¹C-choline) PET. In some embodiments of any one of the methods provided herein, the subject is one who has undergone conventional imaging or the method includes a step of assessing the subject with conventional imaging.

In one embodiment of any one of the methods provided herein, the subject is one with prostatic lesions, or is suspected of having prostatic lesions. In one embodiment of any one of the methods provided herein, the subject is one with prostate bed lesions, or is suspected of having prostate bed lesions. In one embodiment of any one of the methods provided herein, the subject is one with extra-prostatic metastases, or is suspected of having extra-prostatic metastases. In one embodiment of any one of the methods provided herein, the subject is one with pelvic (e.g., lymph node) metastases, or is suspected of having pelvic (e.g., lymph node) metastases. In one embodiment of any one of the methods provided herein, the subject is one with extra-pelvic (e.g., lymph node) metastases, or is suspected of having extra-pelvic (e.g., lymph node) metastases. In one embodiment of any one of the methods provided herein, the subject is one with distant metastases, or is suspected of having distant metastases. In one embodiment of any one of the methods provided herein, this subject is one with, or is suspected of having, bone, visceral organ, soft tissue, iliac, retroperitoneal para/peri aortic lymph node, limited osseous, thoracic spine or lumbar metastases. In one embodiment of any one of the methods provided herein, the subject is one with nodal metastases.

In some embodiments of such subjects, the subject is one that has undergone radiation therapy, cryotherapy or brachytherapy. In some embodiments of any one of the methods provided herein, the subject is one who has undergone radiation therapy, cryotherapy or brachytherapy or the method includes a step of treating the subject with radiation therapy, cryotherapy or brachytherapy.

In one embodiment of any one of the methods provided herein, a subject has had prior antiandrogen therapy, such as with abiraterone. In another embodiment of any one of the methods provided herein, such subject has had prior antiandrogen therapy, such as with abiraterone, but not prior cytotoxic chemotherapy, such as with taxane chemotherapy. In another embodiment of any one of the methods provided herein, any one of such subjects has prostate cancer that has progressed despite these prior treatment(s). In another embodiment of any one of the methods provided herein, any one of such subjects is one with mCRPC that has progressed despite prior treatment(s).

In one embodiment of any one of the methods provided herein, a subject has had multiple rounds of prior antiandrogen therapy, such as with abiraterone. In another embodiment of any one of the methods provided herein, a subject has had prior antiandrogen therapy, such as with abiraterone, but not prior cytotoxic chemotherapy, such as with taxane chemotherapy. In another embodiment of any one of the methods provided herein, any one of such subjects has prostate cancer that has progressed despite the prior treatment(s). In another embodiment of any one of the methods provided herein, any one of such subjects is one with mCRPC that has progressed despite prior treatment(s).

An “antiandrogen,” as used herein, refers to an agent that blocks (e.g., inhibits) the action of androgen hormones and androgen-regulated molecules. Adrenergic receptor antagonists are herein considered to be antiandrogens. The term “antiandrogen” includes antiandrogens, antiandrogen analogs, and antiandrogen derivatives. In prostate cancer, antiandrogens block the activity of testosterone, which typically slows prostate cancer growth. In some embodiments, an antiandrogen blocks enzyme cytochrome P450 17A1, encoded by the CYP17A gene. Antiandrogens may be steroidal or non-steroidal (also referred to as “pure”). Examples of antiandrogens include, without limitation, abiraterone (ZYTIGA®), enzalutamide (XTANDI®), nilutamide (NILANDRON®), flutamide (EULEXIN®), bicalutamide (CASODEX®), orteronel (TAK-700, Tokai Pharmaceuticals, Inc.), apalutamide (Janssen) and darolutatmide (Bayer).

“Progression”, as used herein, refers to prostate cancer cell proliferation that is not reduced, such as with treatment, such as with any one of the prior treatments or combinations thereof that are referred to herein, respectively. Disease progression may be indicated by rising PSA levels (e.g., an increase from baseline or a prior measurement of ≥25% and ≥2 ng/mL above nadir with or without a second such assessment of progression ≥3 weeks later), soft tissue disease progression as defined by RECIST 1.1, bone disease progression defined by two or more new lesions on bone scan, and/or new pain in an area of radiographically evident disease. In one embodiment of any one of the methods provided herein, prostate cancer that is progressing is not substantially inhibited by the prior treatment or combination thereof and would be considered non-responsive by a clinician.

In one embodiment of any one of the methods provided herein, the subject has or has had soft tissue or bone progression, such as with a scan that shows progression relative to a comparison scan performed during prior abiraterone therapy or after discontinuation from abiraterone. In one embodiment of any one of the methods provided herein, the subject has or has had soft tissue or bone progression, such as with a scan that shows progression relative to results from a previous scan, such as performed during prior abiraterone therapy or after discontinuation from abiraterone.

### EXAMPLES

### Example 1—A PrOspective Phase 2/3 Multi-Center Study of ¹⁸F-DCFPyL PET/CT Imaging in Patients with PRostate Cancer—Examination of Diagnostic AccuracY (OSPREY)

### BACKGROUND

Prostate-specific membrane antigen (PSMA) is a transmembrane protein that is overexpressed by prostate cancer (PCa) cells and can be targeted using the novel PET radiotracer ¹⁸F-DCFPyL. This example was aimed to determine the diagnostic performance of ¹⁸F-DCFPyL PET/CT for detecting pelvic lymph node metastases and other distant sites of disease in men with PCa.

### Methods

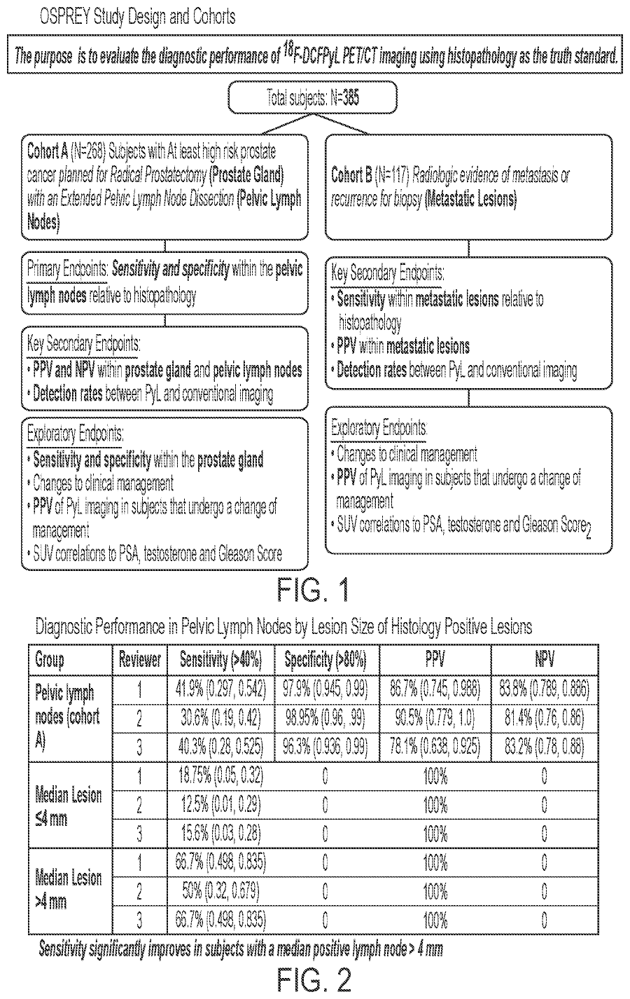

¹⁸F-DCFPyL PET/CT was evaluated in 385 men with high-risk PCa planned for radical prostatectomy and lymphadenectomy (Cohort A, n=268) or radiological evidence of metastatic PCa feasible for biopsy (Cohort B, n=117). 9 mCi (333 MBq) of ¹⁸F-DCFPyL was administered 1-2 hours prior to PET/CT. The coprimary endpoints of sensitivity and specificity of ¹⁸F-DCFPyL PET/CT for detecting pelvic lymph node metastases were evaluated in Cohort A. Secondary endpoints included safety in both cohorts, positive predictive value (PPV) and negative predictive value (NPV) in Cohort A, and sensitivity and PPV in Cohort B. Three central, blinded, and independent readers evaluated the ¹⁸F-DCFPyL scans. Histopathology served as the reference standard to which imaging findings were compared. FIG. 1 provides information regarding the study design and cohorts.

### Results

In Cohort A (n=252 evaluable), the sensitivity of ¹⁸F-DCFPyL PET/CT ranged among the three readers from 30.6-41.9% (lower bound of 95% CI: 19.2-29.7%), with a range of specificities of 96.3-98.9% (lower bound of 95% CI: 93.6-96.0%). Additionally, the PPV and NPV ranged from 78.1-90.5% and 81.4-83.8%, respectively. When the analysis was restricted to median lymph node metastases >4 mm, there was a marked improvement in sensitivity (range 51.7-65.5%; lower bound of 95% CI: 33.5-48.0%). In patients with distant metastatic PCa (Cohort B, n=93 evaluable), the values of sensitivity and PPV ranged from 92.9-98.6% (lower bound of 95% CI: 84.0-91.6%) and 81.2-87.8%, respectively. No drug-related serious adverse events were observed and 27 (7.0%) men experienced ≥1 drug-related adverse event, with dysgeusia (2.1%) and headache (2.1%) being most common.

### Diagnostic Performance Summary (Evaluable Set)

Pelvic Lymph Node Diagnostic Summary
The first co-primary endpoint of specificity ranged from 96-99% and was statistically significant at the pre-specified limit of 80%
The second co-primary endpoint of sensitivity ranged from 31-42% and did not meet the pre-specified confidence limit of 40%
The high PPV (78-91%) and NPV (81-84%) performance demonstrate strong clinical utility of ¹⁸F-DCFPyL PET/CT giving physicians the confidence to rely on the test result

Metastatic Diagnostic Summary
Secondary efficacy endpoint that represents recurrent or metastatic disease outside the pelvis (e.g., bone, soft tissue, and lymph nodes)
In this patient population, high sensitivity (93-99%) and PPV (81-88%) offers strong clinical utility to accurately detect distant metastatic lesions

Extra-Prostatic Diagnostic Summary
The performance of ¹⁸F-DCFPyL to detect extra-prostatic lesions, including the pelvic lymph nodes from extended pelvic lymph node dissection and distant metastatic or recurrent lesions from targeted biopsy, were statistically significant across all diagnostic parameters
This pooled analysis improves on the overall sensitivity of PyL to detect metastatic lesions (both pelvic and distant)

Cohort A and B

Prostate Gland Summary
This is an exploratory endpoint that demonstrates PyL is detecting prostate cancer with near perfect sensitivity and PPV
Confirming these are high risk prostate cancer patients
   Change of Medical Management of Subject Care (defined as, for example, change in one or more of the following):

TABLE 1
Parameter

```
Any Change in Management
  Yes
  No
Change in Diagnostic biopsy per standard of care
  Yes
  No
Change in surgery or type of Surgery Planned
  Yes
  No
Change in planned radiation
  Yes
    Change in Dose of radiation
    Change in Location of radiation
  No
Change in planned chemotherapy based on:
  Yes
    Change in Dose of chemotherapy
    Change in Regimen of chemotherapy
  No
Change in planned hormonal/ADT therapy
  Yes
    Change in Dose of hormonal/ADT therapy
    Change in Regimen of hormonal/ADT therapy
  No
```

### Change in Management Based on Sequential Review of Centrally Read Pre-PyL Imaging and Clinical Characteristics Followed by Re-Review Based on PyL Imaging Results

In one example, a change of management appeared to be indicated in ˜43% of subjects after reviewing the PyL PET/CT image with the most significant change to planned hormonal therapy

Cohort A

### CONCLUSIONS

¹⁸F-DCFPyL PET/CT was well tolerated and demonstrated high overall diagnostic performance in the detection of pelvic lymph node metastases and other distant sites of metastatic disease, as evidenced by its high specificity and PPV. These data suggest that ¹⁸F-DCFPyL PET/CT can enable more accurately informed treatment choices in men with prostate cancer.

### Example 2—Study of ¹⁸F-DCFPyL PET/CT Imaging in Patients with Suspected Recurrence of Prostate Cancer (CONDOR)

TABLE 2
Arms and Interventions

| Arms | Assigned interventions |
|---|---|
| Experimental: 18F-DCFPyL | Drug: 18F-DCFPyL |
| Injection 9 mCi (333 MBq) IV | A single dose of 9 mCi (333 MBq) IV |
| injection of 18F-DCFPyL | injection of 18F-DCFPyL |
| | Other Names: |
| | PyL |

### Outcome Measures

Primary Outcome Measure:

1. Correct Localization Rate (CLR), defined as percentage of subjects with a one-to-one correspondence between localization of at least one lesion identified on ¹⁸F-DCFPyL PET/CT imaging and the composite truth standard, defined as either evaluable histopathology, informative correlative imaging, or PSA response after radiation therapy.

Within 60 days following PyL PET/CT imaging, either biopsy/surgery, conventional imaging, or locoregional radiation therapy of the PyL-suspected lesion(s) will be performed.

Secondary Outcome Measure:

2. Percentage of subjects with a change in intended prostate cancer treatment plans due to ¹⁸F-DCFPyL PET/CT imaging results.

The change in intended prostate cancer treatment plan will be based on Medical Management Questionnaires completed prior to and after PyL PET/CT imaging.

### Eligibility

Minimum Age: 18 Years

Maximum Age:

Sex: Male

Gender Based: Yes-only males subjects will be enrolled in this study.

Accepts Healthy Volunteers: No

Criteria: Inclusion Criteria:
Male >/=18 years of age
Histopathologically confirmed prostate adenocarcinoma per original diagnosis, with subsequent definitive therapy
Suspected recurrence of prostate cancer based on rising PSA after definitive therapy on the basis of:
a. Post-radical prostatectomy: Detectable or rising PSA that is ≥0.2 ng/mL with a confirmatory PSA ≥0.2 ng/mL (American Urological Association [AUA]); or
b. Post-radiation therapy, cryotherapy, or brachytherapy: Increase in PSA level that is elevated by ≥2 ng/mL above the nadir (American Society for Therapeutic Radiology and Oncology [ASTRO]-Phoenix)
Negative or equivocal findings for prostate cancer on conventional imaging performed as part of standard of care workup within 60 days prior to Day 1
Conventional imaging findings per institutional standard of care (SOC scan(s) (e.g., pelvic CT/MRI, whole-body bone scan, NaF, fluciclovine or choline PET) completed within 60 days prior to Day 1. SOC scan(s) performed more than 60 days prior to Day 1 may be repeated as a study Screening procedure and reviewed by the investigator prior to Day 1.
Life expectancy ≥6 months as determined by the investigator
Able and willing to provide informed consent and comply with protocol requirements

### Exclusion Criteria:

Subjects administered any high energy (>300 KeV) gamma-emitting radioisotope within five (5) physical half-lives prior to Day 1
Ongoing treatment with any systemic therapy (e.g., ADT, antiandrogen, GnRH, LHRH agonist or antagonist) for prostate cancer
Treatment with ADT in the past 3 months of Day 1
Receipt of investigational therapy for prostate cancer within 60 days of Day 1
Subjects with any medical condition or other circumstances that, in the opinion of the investigator, compromise the safety or compliance of the subject to produce reliable data or completing the study.

### Example 3—Results from the OSPREY Trial: A PrOspective Phase 2/3 Multi-Center Study of ¹⁸F-DCFPyL PET/CT Imaging in Patients with PRostate Cancer—Examination of Diagnostic AccuracY

### BACKGROUND

Prostate-specific membrane antigen (PSMA) is a transmembrane protein that is overexpressed by prostate cancer (PCa) cells. PSMA-based imaging is considered highly promising for PCa detection and can be targeted using the novel PET radiotracer ¹⁸F-DCFPyL. This prospective multicenter trial was designed to determine the safety and diagnostic performance of ¹⁸F-DCFPyL PET/CT for detecting pelvic lymph node metastases and sites of distant metastases in men with PCa.

### Methods

¹⁸F-DCFPyL PET/CT was evaluated in 385 men with high-risk PCa who were planned for radical prostatectomy with lymphadenectomy (Cohort A) or with radiological evidence of recurrent or metastatic PCa who were planned for biopsy (Cohort B). 9 mCi (333 MBq) of ¹⁸F-DCFPyL was administered 1-2 hours prior to PET/CT. Co-primary endpoints of specificity and sensitivity of ¹⁸F-DCFPyL PET/CT for detecting prostate cancer metastases in pelvic lymph nodes were evaluated in Cohort A. Key secondary endpoints included incidence of adverse events, positive predictive value (PPV) and negative predictive value (NPV) of ¹⁸F-DCFPyL PET/CT imaging to detect pelvic lymph node metastases in Cohort A, sensitivity and PPV of ¹⁸F-DCFPyL PET/CT imaging to detect prostate cancer within sites of metastasis or recurrence in Cohort B. Three central, blinded, and independent readers evaluated the ¹⁸F-DCFPyL scans. Imaging findings were compared to histopathology as the truth standard.

### Results

For detection of pelvic lymph node metastases in Cohort A (n=252 evaluable), ¹⁸F-DCFPyL PET/CT imaging had a median sensitivity of 40.3% (95% CI: 28.1-52.5%), median specificity of 97.9% (95% CI: 94.5-99.4%); and median PPV and NPV of 86.7% (95% CI: 69.7-95.0%) and 83.2% (95% CI: 78.0-88.0%), respectively. In Cohort B (n=93 evaluable), median sensitivity and PPV were 95.8% (95% CI: 87.8-99.0%) and 81.9% (95% CI: 73.7-90.2%), respectively. Sensitivity and PPV of ¹⁸F-DCFPyL PET/CT were evaluated in different lesion locations on a region level (prostatic, pelvic and extra-pelvic) in Cohort B. Median sensitivity and PPV for the pelvic region were 100% (95% CI: N/A) and 79.5% (95% CI: 67.0-92.0%), respectively; and median sensitivity and PPV for the extra-pelvic region were 94.9% (95% CI: 82.0-99.0%) and 86.1% (95% CI: 76.0-96.0%), respectively. There were no evaluable subjects with a local recurrence to the prostatic region for analysis. Individual reader assessments are shown in the table below. No drug-related serious adverse events were observed. Twenty-seven (7.0%) men experienced ≥1 drug-related adverse event, with dysgeusia (2.1%) and headache (2.1%) being most frequent.

### CONCLUSIONS

¹⁸F-DCFPyL PET/CT has a favorable toxicity profile and is generally well tolerated in patients with PCa. ¹⁸F-DCFPyL PET/CT demonstrated high sensitivity in reliably detecting distant metastatic prostate cancer and high specificity in confirming the absence of pelvic lymph node metastases. The associated strong PPV and NPV of ¹⁸F-DCFPyL imaging in these disease settings indicate its potentially high clinical utility.

TABLE 3
Diagnostic Performance by Region (Cohort B)

| Region | Reviewer | Sensitivity | | PPV |
|---|---|---|---|---|
| Prostate bed | — | — | | — |
| Pelvic | 1 | 100% | | 76.2% (0.63, 0.89) |
| | 2 | 96.9% | (0.82, 1.0) | 79.5% (0.67, 0.92) |
| | 3 | 100% | | 88.9% (0.78, 0.99) |
| Extra-pelvic | 1 | 97.4% | (0.85, 1.0) | 86.1% (0.76, 0.96) |
| | 2 | 94.9% | (0.88, 0.99) | 84.1% (0.73, 0.95) |
| | 3 | 86.8% | (0.72, 0.95) | 86.8% (0.76, 0.97) |

### Example 4—Diagnostic Performance of ¹⁸F-DCFPyL in the OSPREY Trial: A Prospective Phase 2/3 Multi-Center Study of ¹⁸F-DCFPyL PET/CT Imaging in Patients (Pts) with Known or Suspected Metastatic Prostate Cancer (mPC)

### BACKGROUND

Accurate detection of prostate cancer is imperative to patient management, yet standard imaging methods perform poorly in accurately detecting mPC. ¹⁸F-DCFPyL is a novel PET imaging agent that selectively binds to prostate-specific membrane antigen, a recognized target for prostate cancer. OSPREY was a prospective, multicenter study in pts with either newly diagnosed high-risk prostate cancer (cohort A), or known or suspected mPC (cohort B). Here we focus on Cohort B.

### Methods

117 men planned for biopsy of recurrent or mPC received ¹⁸F-DCFPyL. Pts underwent image-guided biopsy. Sensitivity, positive predictive value (PPV), and safety of ¹⁸F-DCFPyL PET/CT were the key endpoints for Cohort B. ¹⁸F-DCFPyL PET/CT scans were evaluated by three independent, blinded central readers; and results were compared to histopathology as the truth standard.

### Results

The sensitivity and PPV of ¹⁸F-DCFPyL PET/CT as compared to histopathology ranged from 92.9-98.6% (lower bound of 95% CI: 84.0-91.6%) and 81.2-87.8%, respectively. Diagnostic performance by anatomic location showed high sensitivity and high PPV in all sites of disease (see table below). Only two (1.7%) cohort B pts experienced ≥1 drug-related AE (dysgeusia and generalized rash), both were mild (Grade 1) in severity.

### CONCLUSIONS

¹⁸F-DCFPyL PET/CT was well tolerated and demonstrated high sensitivity and PPV in accurately detecting nodal, bone, and visceral/soft tissue metastases. A positive ¹⁸F-DCFPyL PET/CT scan can represent pathologically proven distant disease, demonstrating ¹⁸F-DCFPyL as a PET imaging agent to favorably influence treatment planning.

TABLE 4

| | | Sensitivity | | PPV | |
|---|---|---|---|---|---|
| | | Range for 3 | | Range for 3 | |
| | N | readers (%) | | readers (%) | |
| Anatomic | Evalu- | (lower bound of | | (lower bound of | |
| Location | able | 95% CI (%)) | | 95% CI (%)) | |
| All tissues | 92-93 | 92.9-98.6 | (84.0-91.6) | 81.2-87.8 | (72.9-80.0) |
| Bone | 43-44 | 90.3-96.9 | (74.0-82.9) | 78.9-82.3 | (66.0-69.5) |
| Lymph | 39 | 93.3-100 | (77.6-86.0) | 80.0-90.6 | (66.8-75.0) |
| Node | | | | | |
| Viscera/ | 10 | 88.9-100 | (54.0-65.5) | 90.0-100 | (57.0-62.0) |
| Soft | | | | | |
| Tissue | | | | | |

### Example 5—Diagnostic Performance of PSMA-Targeted ¹⁸F-DCFPyL PET/CT in Men with Biochemically Recurrent Prostate Cancer: Results from the Phase 3, Multicenter CONDOR Study

### BACKGROUND

Currently available imaging modalities are inadequate in reliably localizing or determining the extent of biochemically recurrent (BCR) prostate cancer (PCa), especially in men with low prostate-specific antigen (PSA) levels. Identifying occult disease in men with BCR is important for treatment planning. ¹⁸F-DCFPyL is a novel PET agent for imaging PCa that binds selectively with high affinity to the cell surface protein prostate-specific membrane antigen (PSMA).

### Subject Criteria

Inclusion Criteria

Post-RP: PSA ≥0.2 ng/mL or

Post-RT or cryotherapy: PSA ≥2 ng/mL above nadir

Negative or equivocal imaging per institution's SOC work-up (e,g., whole body bone scan, CT, MRI, ¹⁸F-fluciclovine or 11C-choline PET, ¹⁸F-FDG PET)

Exclusion Criteria

Ongoing treatment with any systemic therapy

Treatment with ADT within 3 months prior to ¹⁸F-DCFPyL administration

### Methods

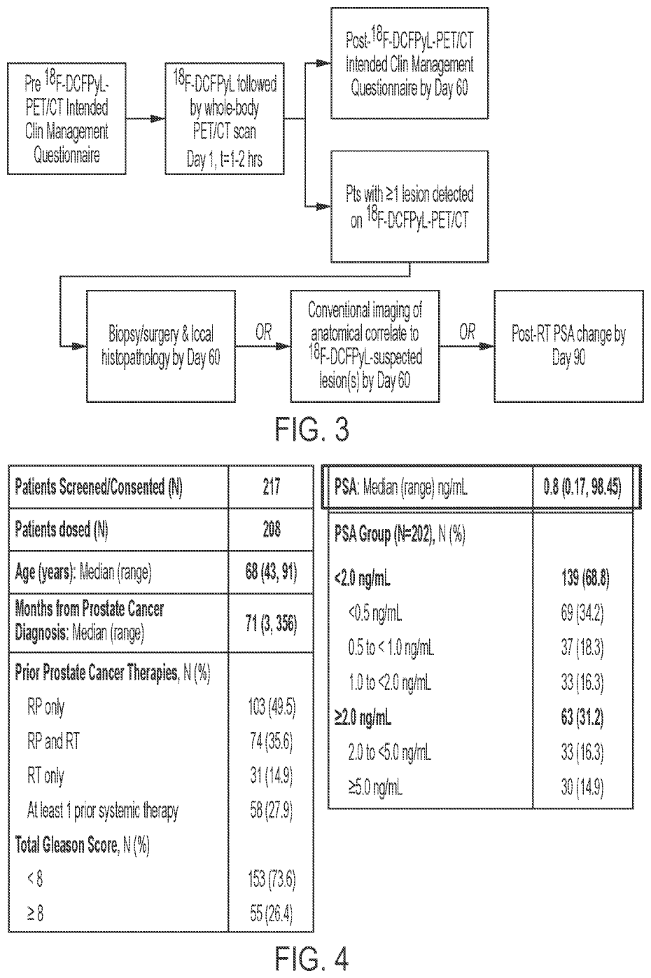

Men ≥18 years of age with a rising PSA after definitive PCa therapy and negative or equivocal imaging were enrolled. A single ˜9 mCi (333 MBq) dose of ¹⁸F-DCFPyL was administered followed by PET/CT from mid-thigh through skull vertex 1-2 hours later. The primary endpoint of this study was the correct localization rate (CLR). CLR is defined as the percentage of patients with a one-to-one correspondence between localization of at least one lesion identified by ¹⁸F-DCFPyL PET/CT and a composite standard of truth (SOT). SOT was determined by two central readers and included either histopathology or another standard imaging modality (i.e., fluciclovine-PET/CT, CT/MRI, bone scan) or alternatively, by a post-treatment PSA change for irradiated lesions. The trial was deemed a success if the lower bound of the 95% confidence interval for CLR exceeded 20% for two of three independent, blinded ¹⁸F-DCFPyL PET/CT reviewers. Overall study design shown in FIG. 3 and select baseline characteristics shown in FIG. 4.

### Results

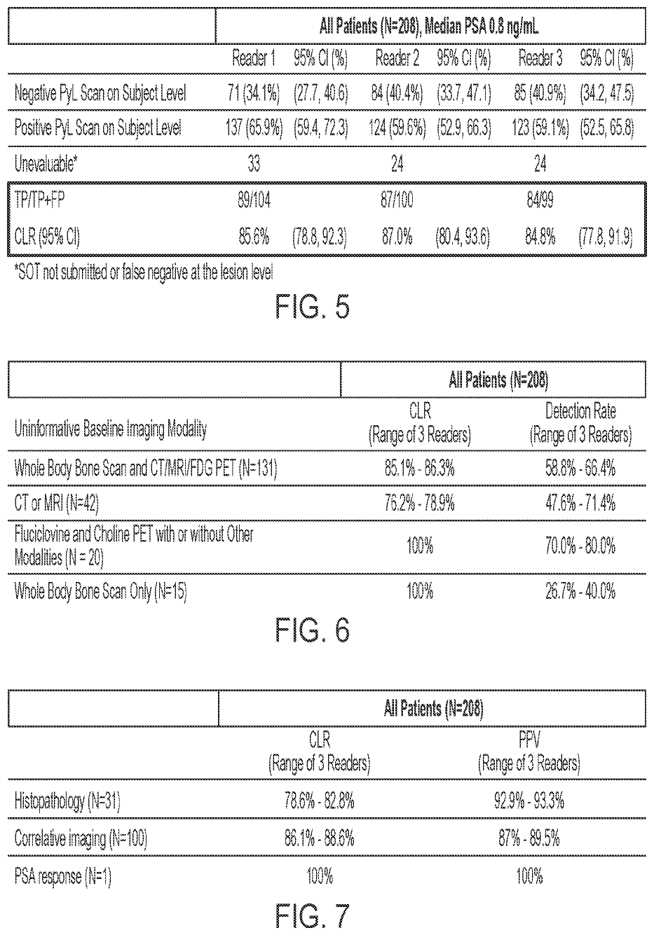

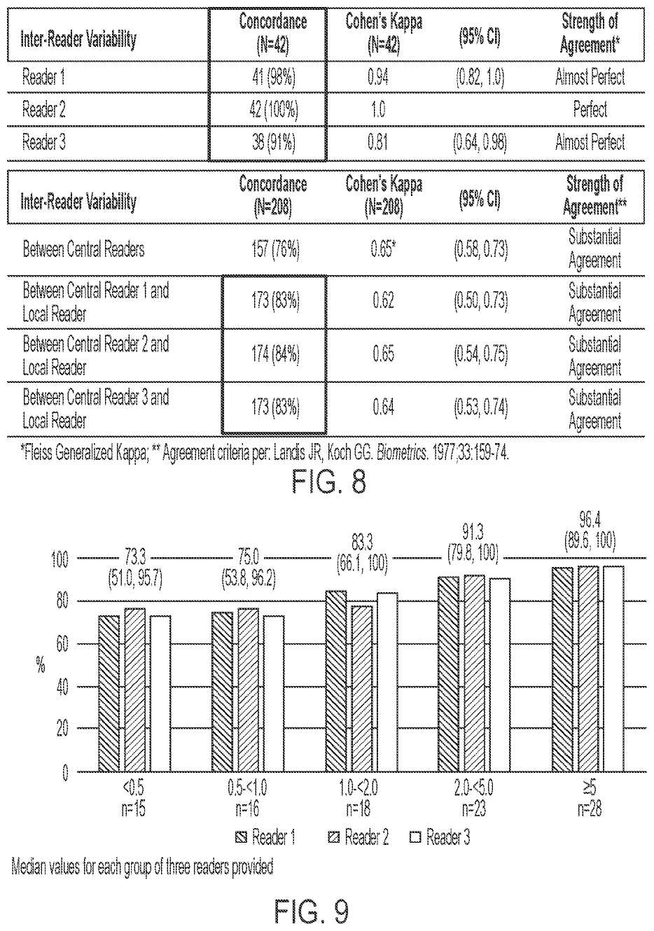

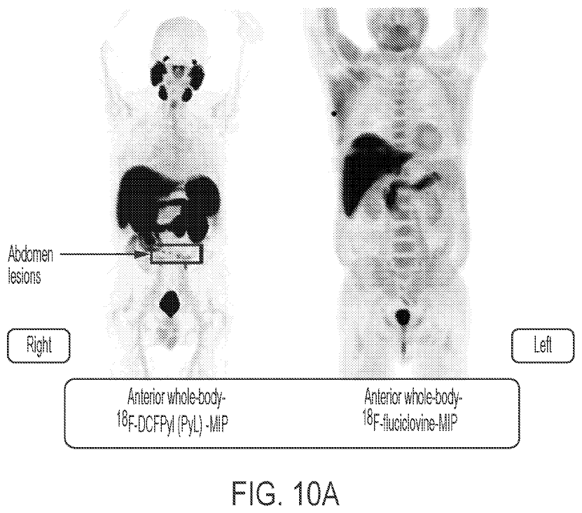

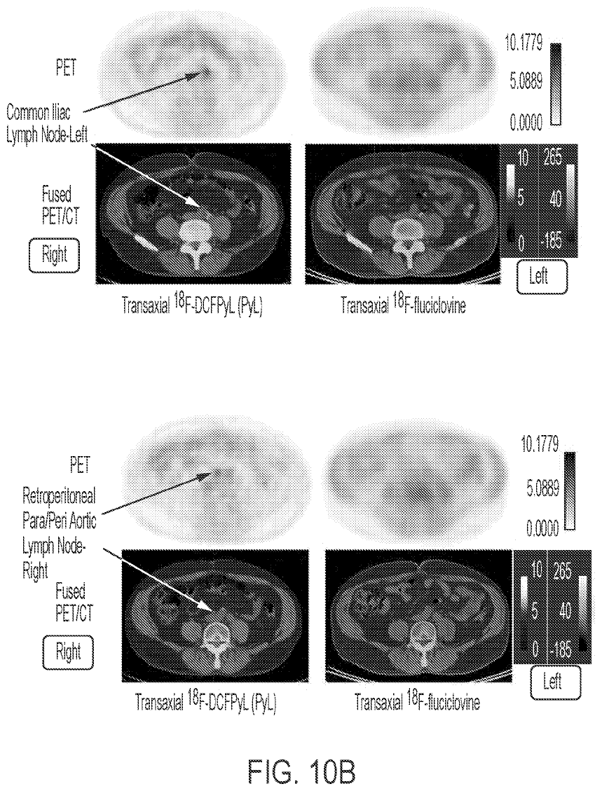

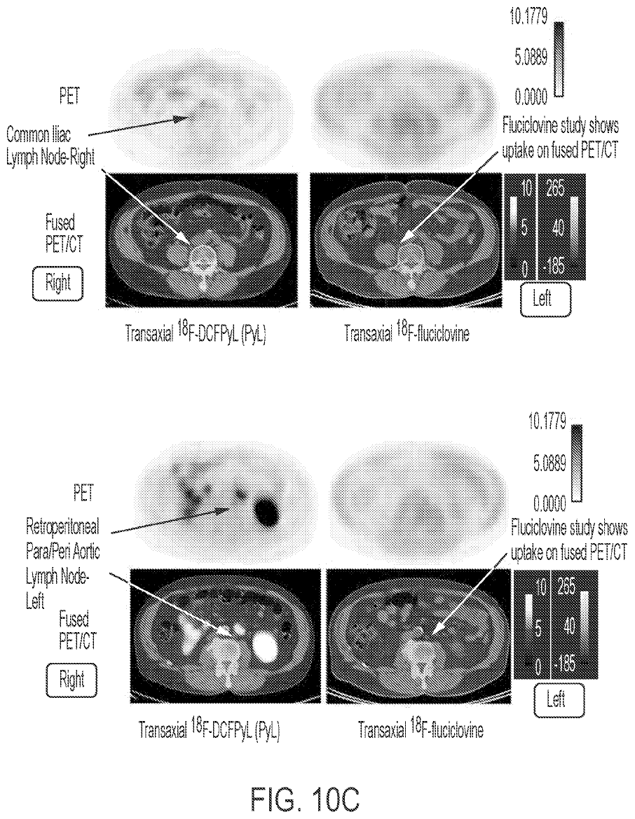

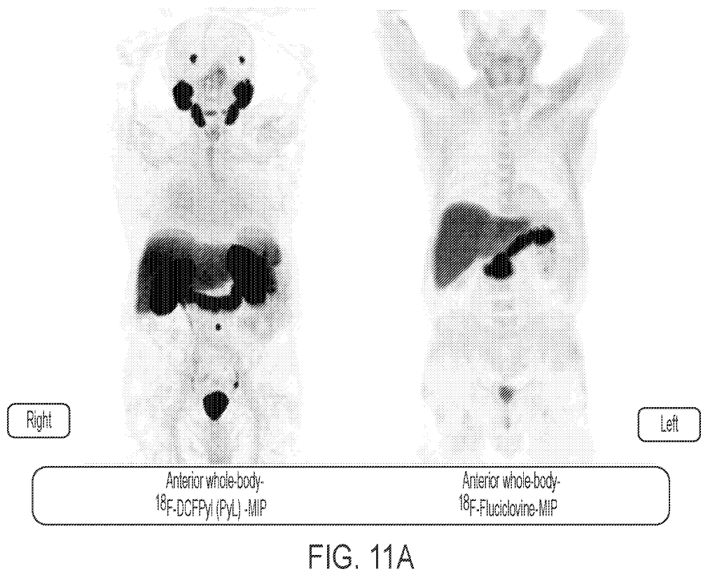

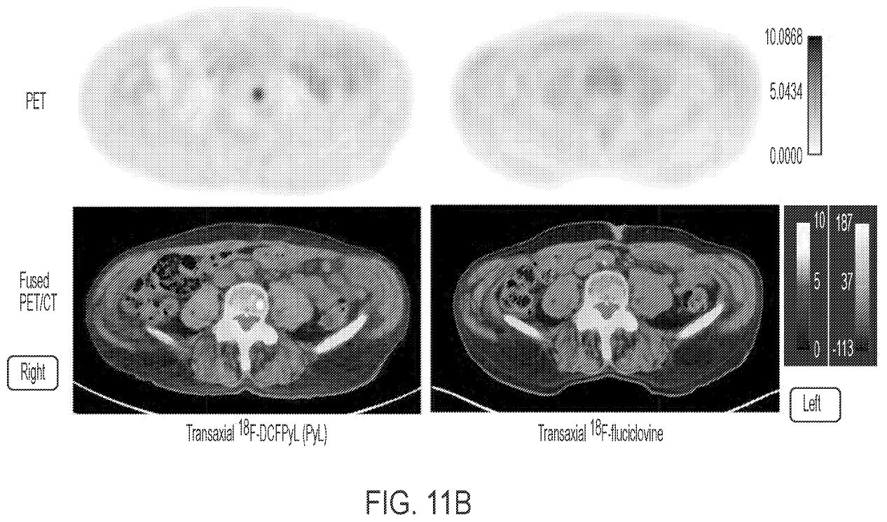

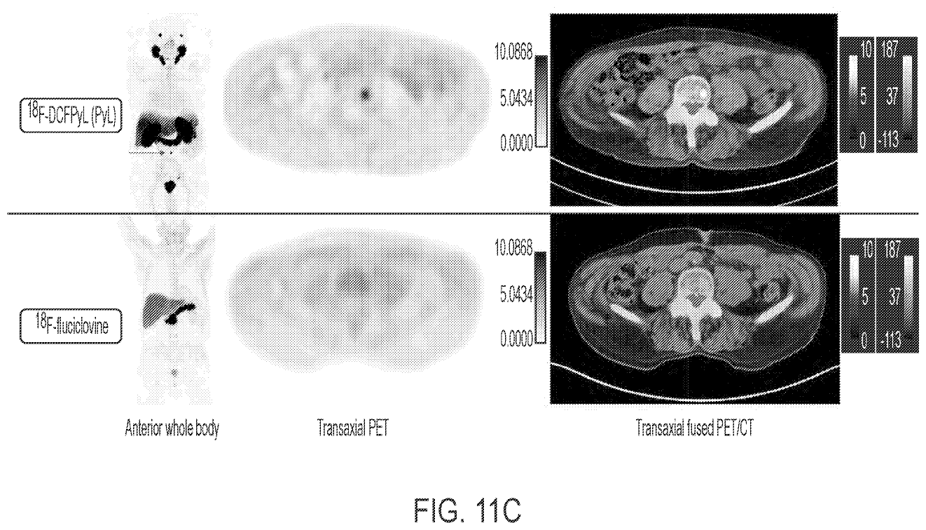

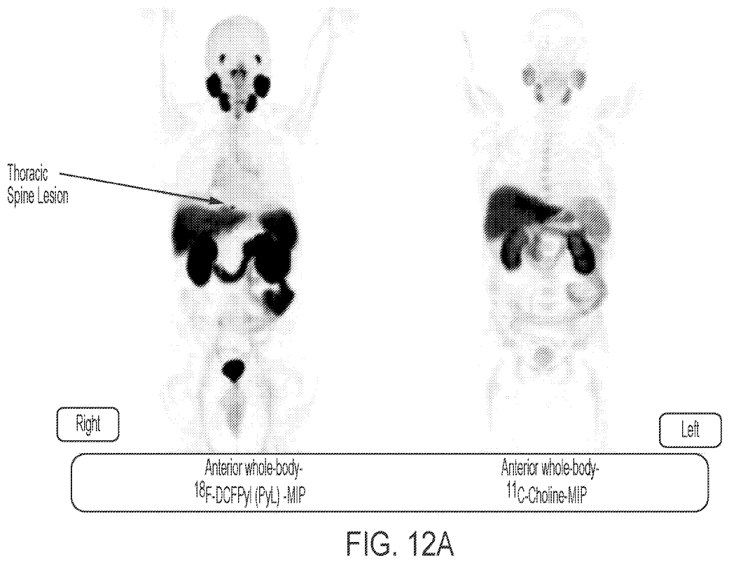

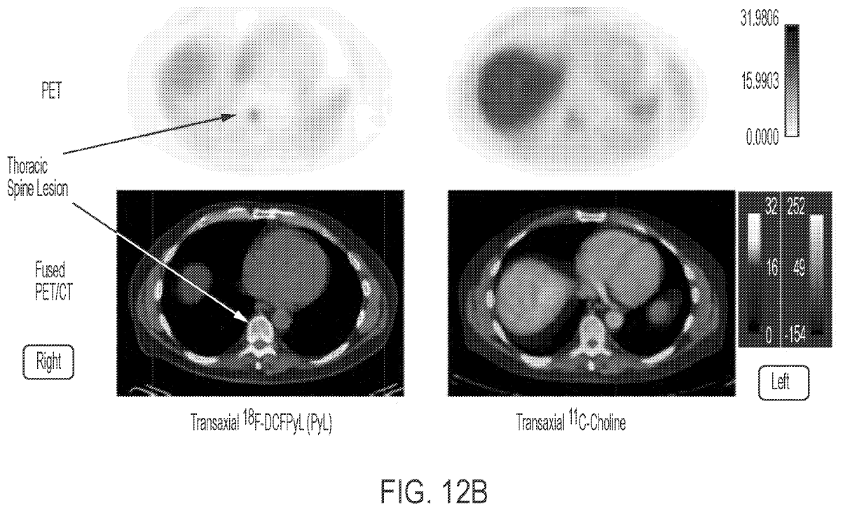

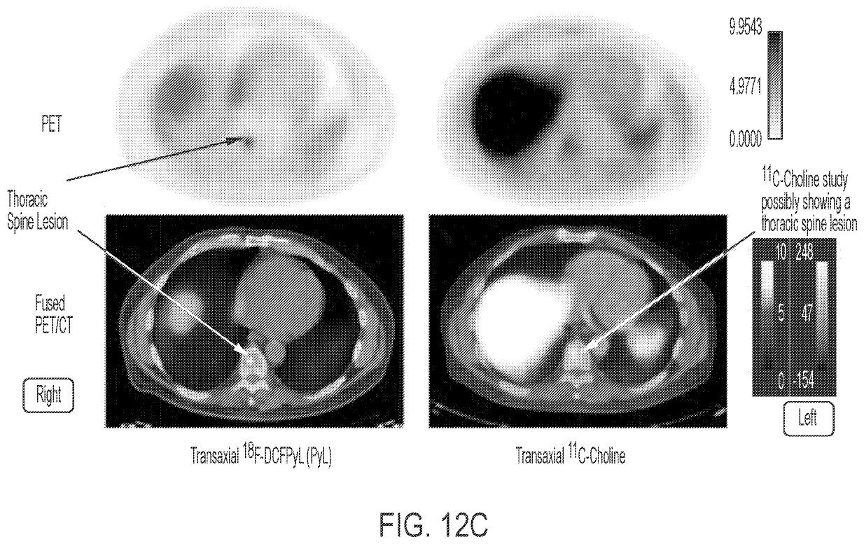

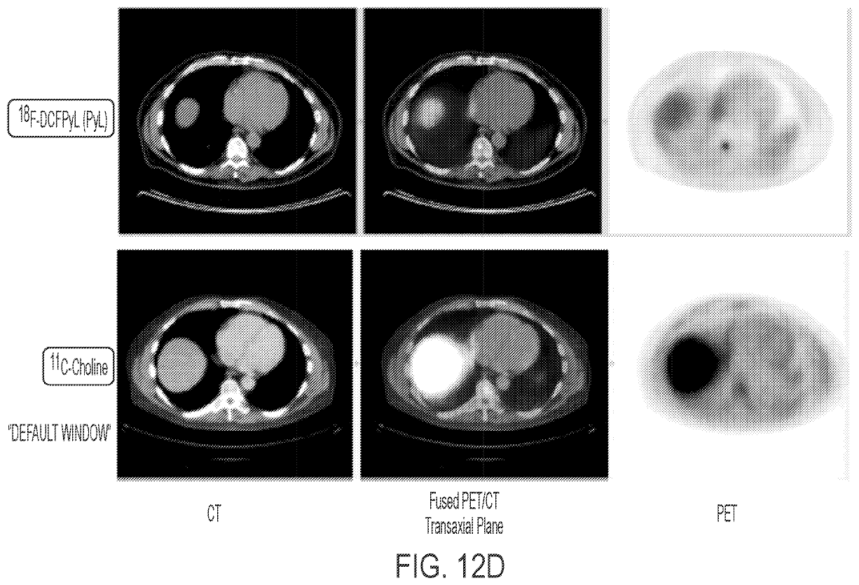

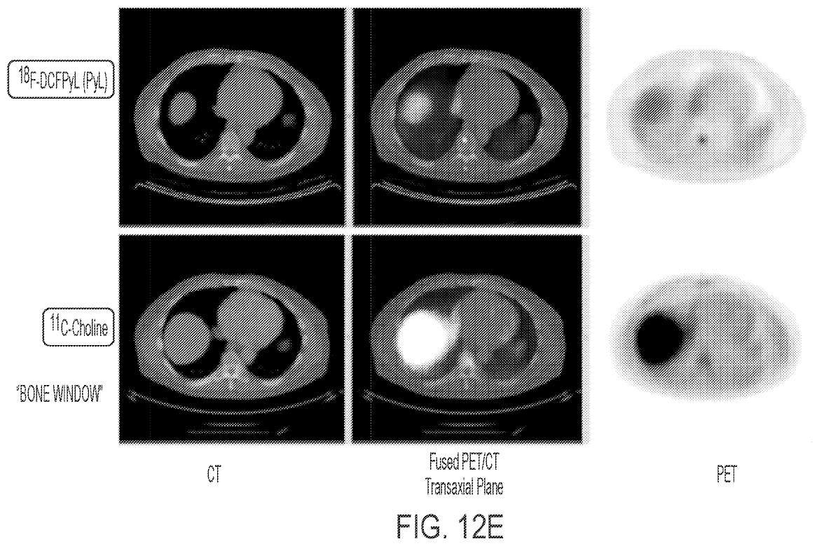

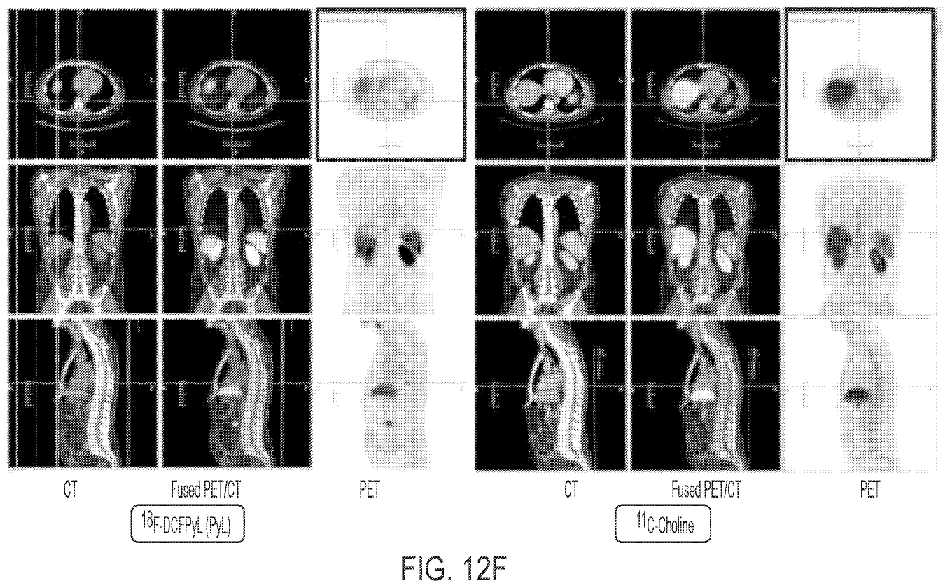

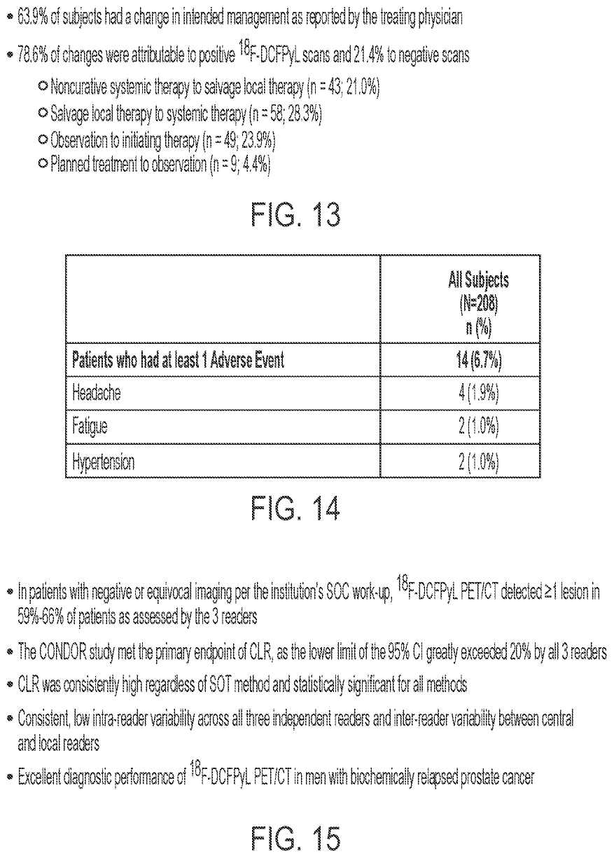

208 men with a median baseline PSA of 0.8 ng/mL (range: 0.2-98.4 ng/mL) underwent ¹⁸F-DCFPyL PET/CT. A total of 59% to 66% of subjects had at least one lesion detected by ¹⁸F-DCFPyL PET/CT by the three blinded independent readers. Among these patients, CLR of 84.8%-87.0% was observed (lower bound of 95% CI: 77.8%-80.4%). A single serious adverse event (AE) was reported as related to study drug (hypersensitivity). The most common AE reported was headache in four patients (1.9%). Results are shown in FIGS. 5-9, 10A-12F, 13, and 15. Safety is outlined in FIG. 14.

### CONCLUSIONS

¹⁸F-DCFPyL PET/CT achieved its primary endpoint with a CLR of up to 87% in localizing recurrent PCa in men with negative or equivocal baseline imaging. These data demonstrate the strong diagnostic performance of PSMA-targeted ¹⁸F-DCFPyL PET/CT and support its use to inform treatment choices in men with recurrent PCa.

### Example 6—A Prospective Phase II/III Multicenter Study of PSMA-Targeted ¹⁸F-DCFPyL PET/CT Imaging in Patients with Prostate Cancer (OSPREY): A Sub-Analysis of Regional and Distant Metastases Detection Rates at Initial Staging by ¹⁸F-DCFPyL PET/CT

### BACKGROUND

Current imaging modalities are suboptimal for the initial staging of men at risk of harboring occult metastatic prostate cancer (PCa). PSMA-based imaging is considered highly promising for PCa detection. ¹⁸F-DCFPyL is a novel PSMA-targeted radiopharmaceutical for positron emission tomography (PET) that may be useful in staging of pts with high risk PCa. Here detection rates and the resulting impact ¹⁸F-DCFPyL can have on staging of patients with high risk PCa are reported.

### Methods

¹⁸F-DCFPyL PET/CT was evaluated in 252 men with high-risk PCa who were planned for radical prostatectomy with lymphadenectomy (RP-PLND). 9 mCi (333 MBq) of ¹⁸F-DCFPyL was administered 1-2 hours prior to PET/CT. Based on TNM staging, ¹⁸F-DCFPyL PET/CT detection rates including lesion counts were systematically analyzed: prostatic (T), pelvic LN (N), extra-pelvic LN (M1a), bone (M1b) & other visceral organs/soft tissue (M1c). Three central, blinded, and independent readers evaluated the ¹⁸F-DCFPyL scans.

### Results

At study entry, 97% and 99% of all evaluable pts had no known nodal or metastatic disease, respectively, based on standard cross-sectional imaging. Of these, ¹⁸F-DCFPyL PET/CT staged 37 (14.7%) pts with N1 disease and 27 (10.7%) pts with M1 disease (1 [0.4%] M1a, 23 [9.1%] M1b, and 3 [1.2%] M1c). In total, 56 (22%) of patients were upstaged to N1 or M1 disease by ¹⁸F-DCFPyL. The positive predictive value of ¹⁸F-DCFPyL based on histopathologic validation for pelvic LNs was 86.7% (95% CI: 70, 95). Only one patient in Cohort A underwent a biopsy of their ¹⁸F-DCFPyL detected M1 finding; histopathology confirmed the metastatic lesion in the spine to be a true positive.

### CONCLUSIONS

A total of 22% of pts with high-risk PCa planned for RP-PLND had regional or distant metastatic lesions detected on ¹⁸F-DCFPyL PET/CT. These results support the utility of ¹⁸F-DCFPyL PET/CT in the staging of men with newly diagnosed high risk PCa to develop optimized treatment paradigms.

### Example 7—Clinical Utility of Preoperative PSMA-Targeted ¹⁸F-DCFPYL PET/CT in Men with High-Risk Prostate Cancer: Diagnostic Performance Comparisons with Pelvic CT or MRI in the OSPREY Prospective, Multi-Center Trial

### INTRODUCTION

Current imaging modalities are suboptimal for the initial staging of men at risk of harboring occult metastatic prostate cancer (PCa) because of the low positive and negative predictive values (PPV and NPV) of pelvic CT/MRI for detecting regional nodal metastases (N1). Prostate-specific membrane antigen (PSMA) is overexpressed by PCa cells and PSMA-based PET has demonstrated high PPV and NPV for identifying regional and distant metastases. ¹⁸F-DCFPyL is a novel PSMA-targeted PET radiopharmaceutical that can improve the accuracy of staging high-risk patients to guide initial therapy planning.

### Methods

¹⁸F-DCFPyL PET/CT was evaluated in 252 men with high-risk PCa due to undergo radical prostatectomy with pelvic lymphadenectomy (RP-PLND). ˜9 mCi (333 MBq) of ¹⁸F-DCFPyL was administered 1-2 hours prior to PET/CT. Sensitivity, specificity, PPV and NPV of ¹⁸F-DCFPyL PET/CT for detecting primary tumor in the gland and pelvic lymph node metastases were compared to conventional imaging (CI), either CT or MRI. Three central, blinded, and independent readers evaluated the ¹⁸F-DCFPyL scans and one blinded central reader interpreted the CI. Imaging results were compared to surgical pathology as the truth standard.

### Results

The diagnostic performance characteristics of ¹⁸F-DCFPyL PET/CT and CI for determining PCa in the gland and pelvic lymph node are shown in the Table below. Despite similar sensitivity (40.3% for ¹⁸F-DCFPyL PET/CT and 41.7% for CI) in detecting N1 disease, the PPV of ¹⁸F-DCFPyL PET/CT (86.7%) was nearly 3-fold higher than CI (29.1%) in identifying true pelvic lymph node metastases. ¹⁸F-DCFPyL-PET/CT was more specific than CI (97.9% vs. 67.3%) and has a higher NPV than CI (83.2% vs. 78.3%). ¹⁸F-DCFPyL-PET/CT was also more sensitive in detecting primary tumor in the prostate gland, with sensitivity of 98% for PyL and 36% for CI).

TABLE 5

| | |
|---|---|
| Conv Imaging¹ | ¹⁸F-DCFPyL PET/CT |
| (Central read; 1 | (Central read; median |
| independent reader) | of 3 independent readers |
| (%, 95% CI) | (%, 95% CI) |

Pelvic Lymph Nodes

| | | |
|---|---|---|
| Sensitivity | 41.7% (0.34, 0.49) | 40.3% (0.28, 0.53) |
| Specificity | 67.3% (0.64, 0.71) | 97.9% (0.95, 0.99) |
| PPV | 29.1% (0.24, 0.35) | 86.7% (0.70, 0.95) |
| NPV | 78.3% (0.75, 0.81) | 83.2% (0.78, 0.88) |

Prostate Gland

| | | |
|---|---|---|
| Sensitivity | 35.9% (0.30, 0.42) | 98.0% (0.96, 1.0) |
| PPV | 100% (0.95, 1.0) | 100% (0.98, 1.0) |

¹Central conventional Imaging reader evaluated a total of 247 scans (CT/MRI) for pelvic lymph node involvement and 245 scans (CT/MRI) for primary tumor in the gland. Only three patients received MRI at baseline.

### CONCLUSION

When compared to pelvic CT or MRI for determining prostate gland and pelvic lymph node disease, using histopathology as the truth standard, the diagnostic performance of ¹⁸F-DCFPyL PET/CT was superior in identifying the true extent of disease and can guide initial therapy in patients with high-risk PCa.

### Example 8—Impact of PSMA-Targeted Imaging with ¹⁸F-DCFPyL-PET/CT on Clinical Management of Patients (Pts) with Biochemically Recurrent (BCR) Prostate Cancer (PCa): Results from a Phase III, Prospective, Multicenter Study (CONDOR)

### BACKGROUND

Current imaging modalities are inadequate for localizing and characterizing occult disease in men with BCR PCa, particularly in patients with low PSAs (<2 ng/mL). ¹⁸F-DCFPyL (PyL) is a novel PET imaging agent that binds selectively with high affinity to PSMA, which is overexpressed in PCa cells. Methods: Men ≥18 years—with rising PSA after definitive therapy and negative or equivocal standard of care imaging (e.g., CT/MRI, bone scintigraphy) were enrolled. A single 9 mCi (333 MBq)±20% dose of PyL was injected, followed by PET/CT 1-2 hours later. Primary endpoint was correct localization rate (CLR), defined as percentage of pts with a 1:1 correspondence between at least one lesion identified by PyL-PET/CT and the composite standard of truth: pathology, correlative imaging, or PSA response. The trial was successful if the lower bound of the 95% confidence interval (LLCI) for CLR exceeded 20% for two of three independent, blinded central PyL-PET/CT reviewers. The secondary endpoint, impact of PyL-PET/CT on clinical management of pts was based on the treating physician's documented clinical plans before and after PyL-PET/CT.

### Results

208 men (median PSA 0.8 [0.2-98.4] ng/mL) underwent PyL PET/CT. The study achieved its primary endpoint: CLR of 84.8% to 87.0% among the three PyL-PET/CT readers; the LLCI for CLR by all three reviewers was >77%. Based on local radiology assessment, PSMA-avid lesion(s) were identified in 69.3% (142/208) of pts. 63.9% (131/205) had a change in intended management after PyL-PET/CT, of which 78.6% (103/131) were attributable to positive PyL finding(s) and 21.4% (28/131) to negative PyL scans. Changes included: salvage local therapy to systemic therapy (n=58); observation before initiating therapy (n=49); noncurative systemic therapy to salvage local therapy (n=43); and planned treatment to observation (n=9). PyL was well tolerated with one drug-related SAE (hypersensitivity) and the most common AE being headache (n=4; 1.9%).

### CONCLUSIONS

PSMA-targeted PyL-PET/CT detected and localized occult disease in most men with BCR presenting with negative or equivocal conventional imaging. PyL-PET/CT led to changed management plans in the majority of patients, thus providing evidence that clinicians find PSMA PET imaging useful in men with recurrent or suspected metastatic PCa.

### Example 9—Diagnostic Performance of ¹⁸F-DCFPyL-PET/CT with Biochemically Recurrent Prostate Cancer: Results from the CONDOR Phase 3, Prospective, Multicenter Study

### BACKGROUND

Current FDA-approved imaging modalities are inadequate for localizing occult disease in men with biochemical recurrence (BCR) of prostate cancer, particularly those with low PSA values. ¹⁸F-DCFPyL is a highly selective, small-molecule PSMA-targeted PET radiotracer. CONDOR (NCT03739684) was a prospective, phase 3 trial designed to determine the performance of ¹⁸F-DCFPyL-PET/CT in patients with BCR and uninformative standard imaging.

### Methods

Men with a PSA ≥0.2 ng/mL after surgery or ≥2 ng/mL above the nadir after radiation therapy were eligible for enrollment. The primary endpoint was the correct localization rate (CLR) defined as positive predictive value with an additional requirement of anatomical lesion colocalization between ¹⁸F-DCFPyL-PET/CT and a composite standard of truth (SOT). The SOT consisted of, in descending priority: 1) histopathology, 2) correlative imaging findings determined by two central readers, or 3) post-radiation PSA response. The trial was considered a success if the lower bound of the 95% confidence interval for CLR exceeded 20% for two of three independent, blinded ¹⁸F-DCFPyL-PET/CT readers. The impact of ¹⁸F-DCFPyL-PET/CT as defined by change in management intent, and safety were evaluated as secondary endpoints.

### Findings

208 men with a median baseline PSA of 0.8 ng/mL (range: 0.2-98.4 ng/mL) underwent ¹⁸F-DCFPyL-PET/CT. The CLR was 84.8%-87.0% (lower bound of 95% CI: 77.8%-80.4%). 63.9% of evaluable patients had a change in intended management after ¹⁸F-DCFPyL-PET/CT. The disease detection rate was 59% to 66% (at least one lesion detected per patient by ¹⁸F-DCFPyL-PET/CT by the central readers).

The performance of ¹⁸F-DCFPyL-PET/CT achieved the study's primary endpoint, demonstrating disease localization even in the setting of negative standard imaging and providing clinically actionable information to clinicians. The data from the CONDOR study further support the use of ¹⁸F-DCFPyL-PET/CT to localize disease in men with recurrent prostate cancer.

### INTRODUCTION

One of the most challenging clinical dilemmas in the management of prostate cancer is the occurrence of a rising prostate specific antigen (PSA) after surgery or radiation therapy in the absence of informative imaging.¹⁻² This condition, known as biochemical recurrence (BCR), declares the presence of persistent or recurrent disease, without the knowledge of its location, and occurs in up to 50% of men who have undergone definitive local therapy.³⁻⁶ The inability to define disease localization is due to the shortcomings of both PSA and standard imaging modalities. PSA is highly sensitive but does not inform on the location or the extent of disease. Conventional anatomic and functional imaging only poorly identify sites of disease in patients with BCR, particularly when PSA values are low.⁷⁻⁹ Novel positron emission tomography (PET) radiotracers, including metabolism-based agents such as ¹¹C-choline and ¹⁸F-fluciclovine, have shown promise, but even their performance degrades with PSA levels <2.0 ng/mL.¹⁰⁻¹² These limitations have stimulated the development of other agents, including radiotracers targeting the cell surface protein prostate-specific membrane antigen (PSMA).¹³⁻¹⁷

¹⁸F-DCFPyL is a small molecule that binds to the extracellular domain of PSMA with high affinity¹⁸ and has shown success evaluating the detection of prostate cancer across a range of disease states, including studies where histopathology served as reference standard.^{13,14,19-21}

CONDOR was designed to demonstrate the performance of ¹⁸F-DCFPyL-PET/CT in men with biochemically recurrent prostate cancer. The primary objective of this study was to evaluate the correct localization rate (CLR) of ¹⁸F-DCFPyL-PET/CT in the detection of recurrent prostate cancer. CLR, a novel endpoint recommended by the United States Food and Drug Administration (FDA), is a measure of positive predictive value (PPV) at the patient level that employed anatomic lesion location matching (co-localization). Secondary objectives were to assess the impact of ¹⁸F-DCFPyL-PET/CT on planned patient management and to evaluate the safety of ¹⁸F-DCFPyL. Exploratory objectives were to determine the detection rate and PPV of ¹⁸F-DCFPyL-PET/CT by region (prostate/prostate bed, pelvis, extra-pelvic regions) and in relation to PSA at the time of BCR.

### Materials and Methods

Trial Design

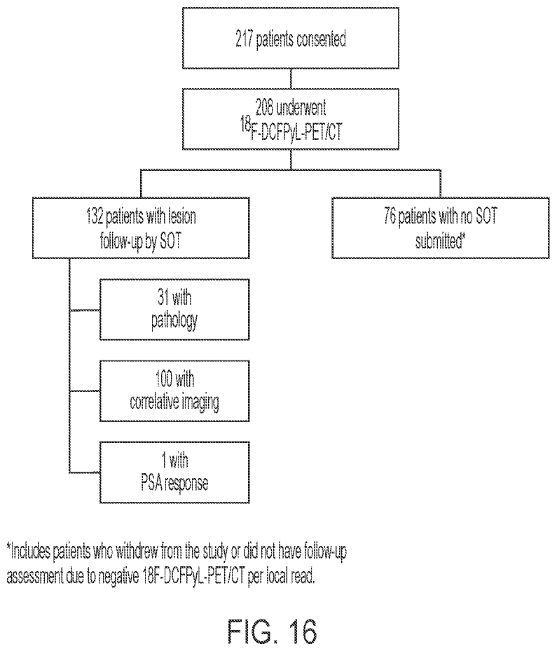

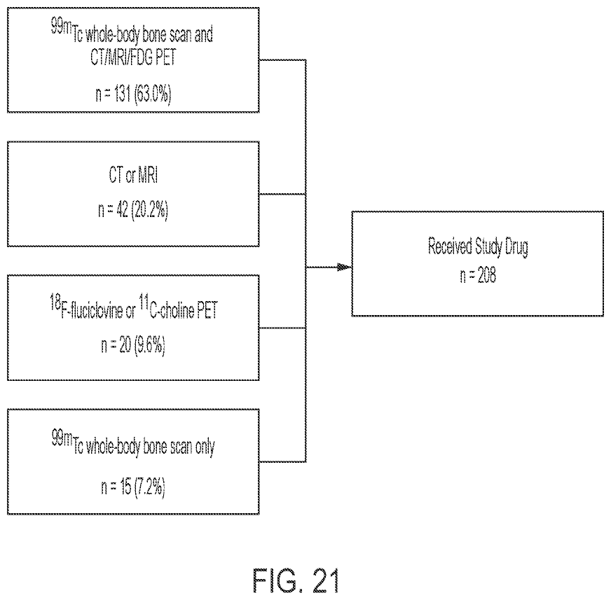

CONDOR (NCT03739684) was a phase 3, prospective, multicenter, open-label, single-arm study designed to evaluate the diagnostic performance and safety of ¹⁸F-DCFPyL-PET/CT in patients with suspected recurrent or metastatic prostate cancer with negative or equivocal conventional imaging (including ¹⁸F-fluciclovine or ¹¹C-choline PET, CT, MRI, and/or whole-body bone scintigraphy) per institutional standard of care (FIG. 21 and Table 11). The Standards for Reporting of Diagnostic Accuracy (STARD) flow diagram is depicted in FIG. 16. The study was conducted across 14 sites in the United States and one site in Canada and was approved by the Institutional Review Board/Research Ethics Board at each participating institution.

Study Population

Men ≥18 years of age with biochemically recurrent adenocarcinoma of the prostate treated with a radical prostatectomy or radiation therapy were eligible for the study. BCR after radical prostatectomy was defined as a rising PSA to ≥0.2 ng/mL.²² For patients treated with radiation therapy, BCR was defined as a post-radiation PSA value of ≥2 ng/mL above a given patient's nadir value.²³ All enrolled patients required negative/equivocal findings for prostate cancer on standard of care imaging performed within 60 days of study drug injection. Prior to study enrollment, a written informed consent was obtained from all patients.

Exclusion criteria included administration of any high energy (>300 KeV) gamma-emitting radioisotope within five physical half-lives prior to study drug injection, and prior androgen deprivation therapy (ADT) within 3 months of imaging, or receipt of investigational therapy for prostate cancer within 60 days of the day of injection and imaging. Ongoing treatment with systemic therapy intended for prostate cancer was prohibited. Patients with any other medical conditions or circumstances that, in the opinion of the investigator, would compromise the safety or compliance of the patient to produce reliable data or completing the study were also excluded.

Screening

Demographic information, baseline characteristics (date of birth, race, ethnicity, height, and weight) and clinically relevant medical history were recorded. The patient's prostate cancer medical history, including American Joint Committee on Cancer (AJCC) stage, Gleason score, pretreatment PSA, and all past/present therapies, were obtained. Standard of care imaging as determined by local practice and obtained within 60 days of administration of ¹⁸F-DCFPyL, was reviewed. This imaging could include standard cross-sectional imaging such as CT or MRI, bone scintigraphy, or other molecular imaging test such as ¹⁸F-fluciclovine or ¹¹C-choline PET. All baseline conventional images were submitted to a central imaging core laboratory for assessment. A blood sample for total PSA was obtained from enrolled patients and analyzed by a central core laboratory.

Medical Management Questionnaire

The treating Investigator completed the pre-PET Medical Management Questionnaire (MMQ) to document the initial intended management plan for the patient based on available clinical information and local conventional imaging results. Within 60 days after ¹⁸F-DCFPyL-PET/CT, the treating Investigator completed the post-PET MMQ to document whether a change to the initial intended management plan was warranted.

¹⁸F-DCFPyL Dosing and PET/CT

The protocol-specified dose of ¹⁸F-DCFPyL was 9 mCi (333 MBq) administered intravenously (IV) 1-2 hours prior to PET/CT. Patients voided prior to imaging, and PET and non-contrast CT images were acquired from the mid-thigh through the skull vertex. All ¹⁸F-DCFPyL-PET/CT scans were submitted to the central imaging core laboratory for assessment. Patients with positive ¹⁸F-DCFPyL-PET/CT scans based on local interpretation were scheduled for post-¹⁸F-DCFPyL-PET/CT follow-up to verify suspected lesion(s) based on a composite standard of truth (SOT). (FIG. 16)

Tiered Composite SOT

Because of the expected absence of an amenable lesion for histologic verification in all patients, a composite SOT was employed based on assessment performed or initiated within 60 days following ¹⁸F-DCFPyL-PET/CT. These reference standards were defined (in descending priority) as (1) evaluable histopathology results from prostatectomy, salvage pelvic lymph node dissection or biopsy; (2) imaging findings using ¹⁸F-fluciclovine or ¹¹C-choline PET, or focused MRI or CT; or (3) if neither of the above was available or informative, confirmed PSA response up to 9 months post-radiation initiation (with no concomitant ADT) of all PET-positive foci. PSA response was defined as PSA decline by ≥50% from baseline that was confirmed on repeat measurement within 4 weeks, based on central laboratory results.

Central Imaging Review

A central imaging core laboratory was employed to independently manage image handling, reader training, reader sessions and data collection. This review consisted of two discrete imaging evaluations:
1) ¹⁸F-DCFPyL-PET/CT assessment was performed by three independent, blinded nuclear medicine readers trained in the interpretation of ¹⁸F-DCFPyL-PET. The readers had no access to any clinical information, including PSA values or other imaging available for a patient. Each reader independently evaluated a patient's ¹⁸F-DCFPyL-PET/CT study according to a one-reader paradigm, without being informed by either of the other two readers and without input from the Truth Panel, the local Investigator, or study sponsor.
2) Each image obtained as part of the SOT was assessed by two independent readers (the Truth Panel), who assessed these images for the presence or absence of prostate cancer. These readers also adjudicated the accuracy of needle placement during image-guided biopsy if biopsy was performed. The Truth Panel members were not members of the central ¹⁸F-DCFPyL-PET reader group and were blinded to all data generated by the central ¹⁸F-DCFPyL-PET/CT readers.

Efficacy Outcomes

The primary endpoint of the study was the CLR of ¹⁸F-DCFPyL-PET/CT. CLR, a novel endpoint recommended by the United States Food and Drug Administration (FDA), is a measure of PPV at the patient level that employed anatomic lesion location matching (co-localization) of the lesion identified by ¹⁸F-DCFPyL-PET/CT central readers and the lesion identified by Truth Panel central readers and/or pathology. CLR was defined as the percentage of patients with a one-to-one correspondence between at least one lesion identified on ¹⁸F-DCFPyL-PET/CT by the central readers and the composite SOT.

The secondary endpoints were the percentage of patients with a change in intended prostate cancer treatment plans after ¹⁸F-DCFPyL PET/CT based on the MMQ that was completed pre-and post-¹⁸F-DCFPyL PET/CT, as well as safety of ¹⁸F-DCFPyL.

Exploratory endpoints were evaluation of detection rates and PPVs of ¹⁸F-DCFPyL PET/CT at the region level (i.e., prostate/prostate bed, pelvis, and extra-pelvic regions) and detection rate of ¹⁸F-DCFPyL PET/CT as a function of baseline PSA (<0.5, 0.5-<1.0, 1.0-<2.0, 2.0-<5.0, or ≥5.0 ng/mL).

Statistical Methods

Determination of sample size is described in the study protocol. The safety and efficacy populations for analysis consisted of all patients who received ¹⁸F-DCFPyL.

Primary Endpoint Analysis

The CLR is computed as 100×TP/(TP+FP), where TP=true positive result and FP=false positive results for each central imaging reader. A TP result is defined as a patient with both a positive lesion(s) on ¹⁸F-DCFPyL PET/CT and a positive result on the composite truth standard within the same anatomic location as defined within the Statistical Analysis Plan. A FP result is defined as a patient with positive lesion(s) on ¹⁸F-DCFPyL PET/CT identified by the central reader with negative findings for prostate cancer according to the composite truth standard. 60% of the imaged patients were expected to have a positive scan, and 30% of the 60% were expected to have a confirmatory SOT that was positive. The two-sided 95% confidence interval (CI) for CLR for each reader was calculated using the normal approximation for a single binomial variable. The success criterion for the primary endpoint was the lower limit of the 95% CI to exceed 20% for at least 2 of the 3 readers.

Secondary Endpoint Analysis

The percentage of patients with a change in intended prostate cancer treatment plan before and after ¹⁸F-DCFPyL-PET/CT was reported with the corresponding two-sided 95% CI using the normal approximation for the binomial variable.

Exploratory Endpoint Analysis

PPV was calculated for patients with positive ¹⁸F-DCFPyL-PET/CT scans as TP/(TP+FP)×100%. Detection rate and PPV by region (i.e., prostate/prostate bed, pelvic, extra-pelvic) and as a function of baseline PSA were analyzed using a two-sided 95% CI presented for each central imaging reviewer and for the local site interpretation separately based on a normal approximation to the binomial distribution.

Safety Outcomes

Safety assessments included monitoring for the incidence of treatment-emergent adverse events (AEs) from the time of ¹⁸F-DCFPyL dosing up to 7±3 days post-dose. The severity grading of AEs was assessed according to the National Cancer Institute Common Terminology Criteria for Adverse Events (NCI CTCAE), Version 4.03. Pre- and post-treatment assessments also included vital signs and concomitant medications/procedures.

### Results

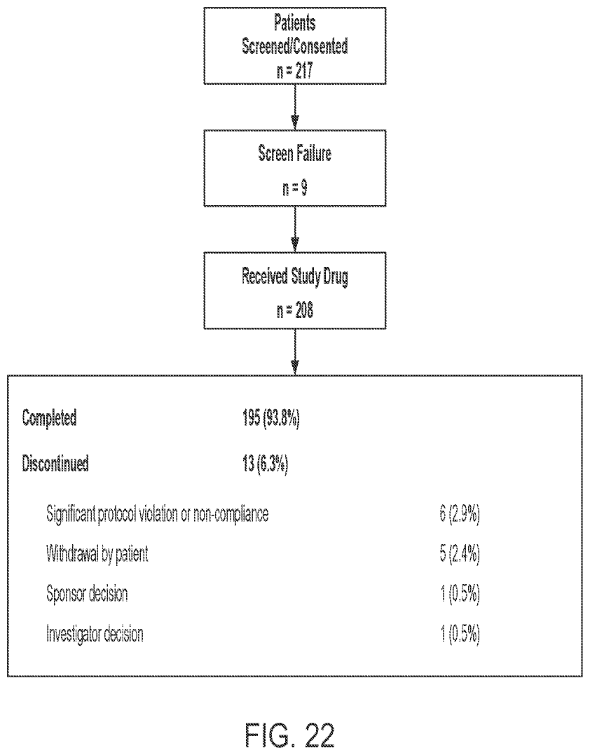

Patients were enrolled across 13 sites in the United States and 1 site in Canada. 217 patients were screened for eligibility; 9 were screen failures and 208 men were enrolled (FIG. 16 and FIG. 22). The median patient age was 68 years (range 43-91), and 67.8% were ≥65 years old. The median baseline PSA level was 0.8 ng/mL (range 0.2-98.4), and the majority of the patients (68.8%) had PSA levels <2.0 ng/mL. The median time from the initial prostate cancer diagnosis was 71 months (range 3-356). Prior treatment was radical prostatectomy in 49.5%, radical prostatectomy and radiation therapy in 35.6%, and radiation alone in 14.9%. Baseline characteristics and PET imaging details are further summarized in Table 6.

¹⁸F-DCFPyL PET/CT detected ≥1 lesion in 59.1% to 65.9% patients as assessed by three independent blinded central readers. The primary endpoint of CLR was met as the lower limit of 95% CI exceeded 20% by all three readers. The CLR ranged from 84.8% to 87.0% among the three independent blinded readers (the lower bound of the 95% CI ranged from 77.8% to 80.4%) (Table 7).

When analyzed for CLR and PPV by SOT based on histopathology, correlative imaging, and PSA response, the CLR was 78.6%-100% across the three SOT methods for all three independent blinded readers (Table 8-10). For the one patient in whom PSA response was the SOT, all three reviewers identified ¹⁸F-DCFPyL PET/CT positive lesions. This patient had a 93% PSA reduction (confirmed) at 3 months post RT.

CLR by Baseline PSA and Detection Rate

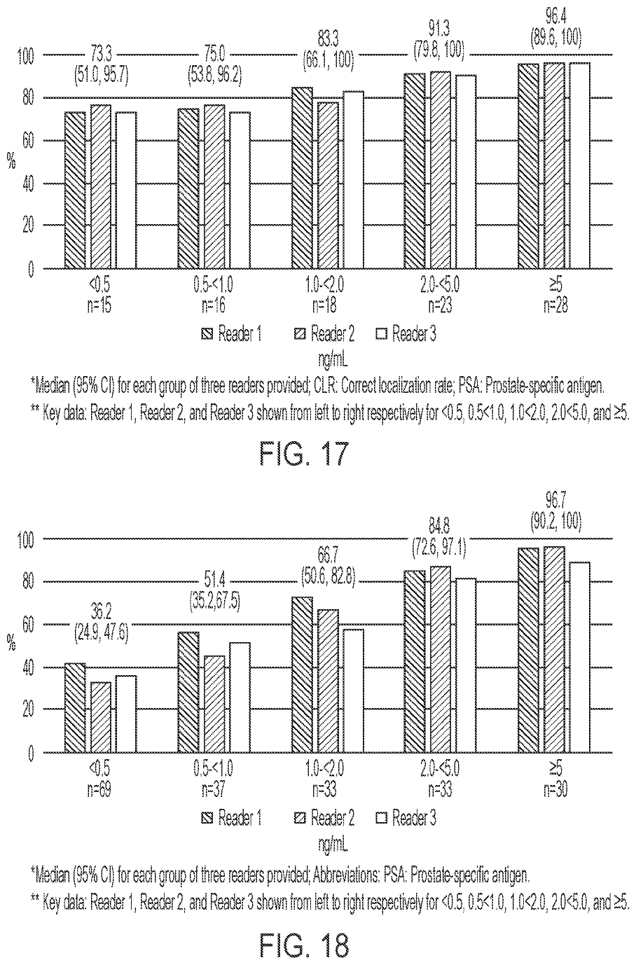

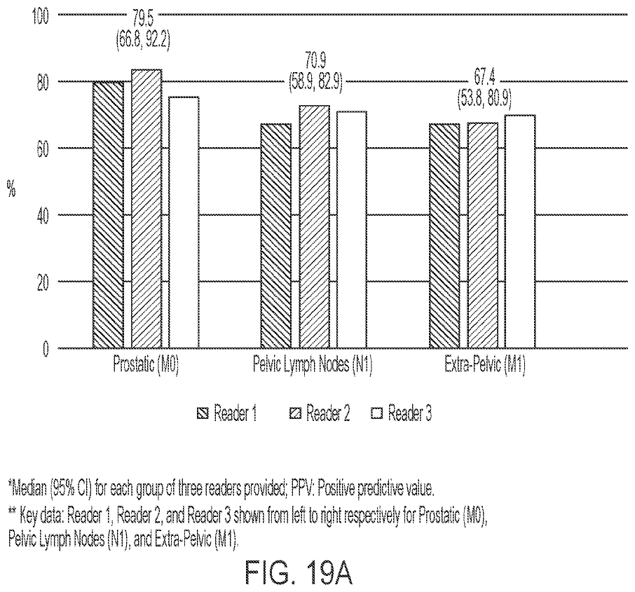

In patients with baseline PSA levels <0.5 ng/mL, the median CLR was 73.3% while patients with a PSA of ≥5 ng/mL had a median CLR of 96.4% (FIG. 17 and Table 12). The lesion detection rate rose with increasing PSA levels with the detection rates ranging from 36.2% (<0.5 ng/mL) to 96.7%. (≥5 ng/mL) (FIG. 18 and Table 13).

Positive Predictive Value by Anatomic Region

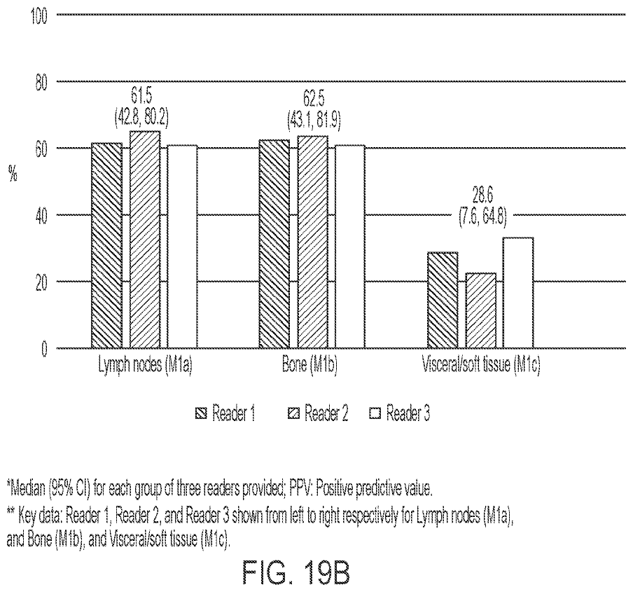

PPV of ¹⁸F-DCFPyL PET/CT was determined in detection of recurrent disease by anatomic regions (prostate/prostate bed, pelvis, and extra-pelvic regions) from the composite SOT in patients with at least one ¹⁸F-DCFPyL-positive lesion. The PPV was consistently high in all anatomic regions. The PPV in the prostatic region ranged between 75.0% and 83.3% among the three independent readers. Similarly, in the pelvic lymph nodes, the PPV was between 67.2% and 72.7%, and in the extra-pelvic regions, it ranged from 67.3% to 69.8%. (FIGS. 19A-19B and Tables 14-15)

Change in Planned Medical Management

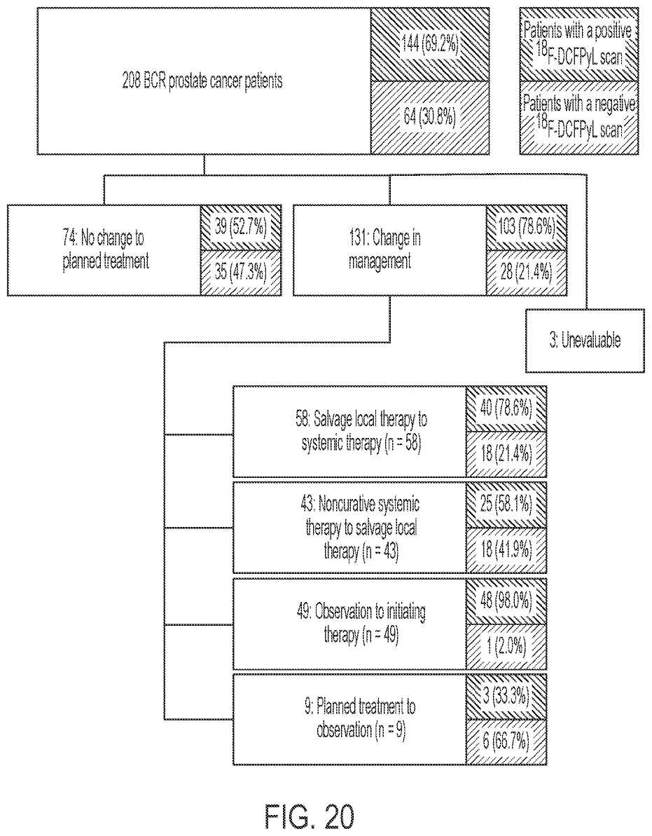

The treating physicians completed pre- and post-¹⁸F-DCFPyL-PET/CT MMQs for 205 patients. Nearly two-thirds (63.9%; n=131) of these patients had a change in intended disease management plan. Of these 131 patients, 103 (78.6%) were associated with positive ¹⁸F-DCFPyL PET/CT findings, and 28 (21.4%) were associated with negative findings. The most frequent changes to treatment management plans after the ¹⁸F-DCFPyL-PET/CT imaging results included salvage local therapy that was either supplemented or replaced by systemic therapy (n=58; 28.3%), observation to initiating therapy (n=49; 23.9%), systemic therapy to salvage local therapy (n=43; 21.0%), and planned treatment to observation (n=9; 4.4%). (FIG. 20 and Table 16)

Safety Results

Fourteen (6.7%) patients experienced 21 AEs, with headache (1.9%), fatigue (1.0%), and hypertension (1.0%) being the most frequent. Only one patient (0.5%) experienced AEs that were Grade 3 in severity and were serious (hypersensitivity, headache, and paresthesia). This patient had a significant history of allergic reactions; and all three SAEs resolved. There were no Grade 4 AEs, nor were there deaths or AEs that led to discontinuation of the study. No clinically relevant changes in vital signs were observed from pre-dosing to post-dosing and concomitant medications and procedures were unremarkable.

### Discussion

The prospective, multicenter, open-label phase 3 CONDOR study was designed to evaluate the performance of ¹⁸F-DCFPyL as a PET radiopharmaceutical in patients with prostate cancer BCR and non-informative standard of care imaging. CONDOR used a rigorous central reader paradigm, robust clinical and data monitoring standards, and a novel primary efficacy endpoint with a composite SOT. The study far exceeded its primary endpoint threshold, demonstrating a high CLR that suggests ¹⁸F-DCFPyL-PET/CT is an accurate method for the detection of sites of disease in men with BCR.

The CONDOR population generally represents BCR patients with low PSA values (median PSA 0.8 ng/mL), when crucial clinical decisions are made as to whether the patient warrants salvage local or metastasis-directed therapy with curative intent, or systemic therapy without curative intent, or some combination of local and systemic treatments. An accurate understanding of the distribution of disease is key to rational treatment planning. In our study population, the PSA was <2.0 ng/mL in 68.8% of patients, <1.0 ng/mL in 52.5% and <0.5 ng/mL in 34.2%. As such, the study provides prospective evidence of diagnostic accuracy to reliably detect prostate cancer recurrence or metastases in patients in whom currently available conventional imaging and approved molecular imaging modalities are suboptimal. Notably, a total of 59.6% to 65.9% of patients had at least one occult lesion detected among the three readers and the CLR was consistently high across all SOT methods, anatomic regions, and in patients with PSA 0.2 to <2.0 ng/mL (>73%). This performance of ¹⁸F-DCFPyL-PET/CT is substantially better than the detection rates and PPVs of ¹⁸F-fluciclovine and ¹¹C-choline PET in patients having PSA values within that range.^{11,12,15}

This study furnishes direct evidence that clinicians can utilize the information contained in the ¹⁸F-DCFPyL scan to revise their treatment plan and goals of care relative to the plan that they had originally made based on non-informative standard imaging. The intended goal of care changed from non-curative using systemic therapy to curative with salvage local therapy in 21% of patients. The intended change from salvage local therapy alone to either supplementation with or replacement by systemic therapy occurred in 28% of patients. Twenty-four percent of patients had a change from observation to initiating therapy, while 4.4% had the converse. Overall, the ability of ¹⁸F-DCFPyL-PET/CT to localize and detect the extent of disease recurrence offers physicians the potential to directly impact treatment management planning, with the goal to ultimately improve treatment outcomes in men with recurrent prostate cancer. This clinically actionable information is provided via an imaging modality that is eminently safe, as well. The evaluation of change in patient management after PET showed that such changes occurred frequently.

While prospective single-center trials with ¹⁸F-DCFPyL-PET imaging in BCR have been recently reported, only Mena et al reported on the PPV as verified by a composite standard of truth.^{6,14,25,26} CONDOR represents the first multi-center prospective trial of ¹⁸F-DCFPyL-PET conducted across 14 centers for the BCR population. By design, the study focused on CLR, which fundamentally is PPV. Consequently, a limitation of this study is that the “truth” of ¹⁸F-DCFPyL scans with no findings at all is unknown. Most cases for which SOT data were not submitted were directly related to the design of the study, because verification of ¹⁸F-DCFPyL-PET/CT results was not required in patients whose scans were negative per local radiology assessment. Trying to find occult disease not detected by PET would have required following these patients without treatment to see if the disease became evident over time; this was not a practical or ethical option. Thus, we cannot determine whether these false-negative cases reflect PSMA-negative disease (which occurs in 5-10% of prostate cancers)²⁷, inexperience of local readers, small volume disease (similar to the poor detection of small nodal deposits in OSPREY Cohort A),²¹ or obscuration of lesions in or adjacent to tissues with high uptake of ¹⁸F-DCFPyL (e.g., liver) or containing excreted tracer (ureters, bladder, urethra). Accordingly, the negative predictive value of ¹⁸F-DCFPyL-PET/CT in this patient population with noninformative standard imaging could not be assessed.

⁶⁸Ga-labelled PSMA PET radiotracers have been used in the academic setting, primarily in large medical centers and with promising results.^{13,28 18}F-labeled PSMA PET imaging agents, in general, can offer important advantages over ⁶⁸Ga agents, including ease of product distribution due to the longer half-life of ¹⁸F, increased production capacity by cyclotron, and better performance given the lower positron energy and higher contrast resolution with ¹⁸F.²⁹⁻³⁰ From a performance standpoint, there are few head-to-head comparisons¹³ and certainly none that contain the rigorous methodology of prospectively defined endpoints, a study population with no informative standard imaging, and independent blinded readers to minimize bias that defined the CONDOR design.

In summary, this prospective multi-center trial, designed to evaluate the performance of ¹⁸F-DCFPyL-PET/CT against a composite SOT in men with BCR of prostate cancer met its primary endpoint of high CLR and also demonstrated that ¹⁸F-DCFPyL-PET/CT was associated with frequent changes in disease management plans. These data support ¹⁸F-DCFPyL-PET/CT as a safe and robust imaging tool to reliably detect recurrent prostate cancer, even at low PSA levels, thus providing new actionable information by the localization of otherwise occult disease. Taken together, the CONDOR and OSPREY pivotal studies demonstrate that ¹⁸F-DCFPyL-PET offers a non-invasive means to detect disease with confidence across the entire spectrum of prostate cancer, from patients with localized high-risk disease to those with metastatic disease, and can be a reliable tool to develop optimized treatment paradigms based on more accurate disease localization.

TABLE 6
Baseline characteristics and ¹⁸F-DCFPyL dosing/uptake time

| Characteristics | n = 208 | |
|---|---|---|
| Age (years): median (range) | 68 | (43, 91) |
| Age ≥65 years, n (%) | 141 | (67.8) |
| Months from prostate cancer | 71 | (3, 356) |
| diagnosis: median (range) | | |
| Prior prostate cancer therapies | | |
| RP only, n (%) | 103 | (49.5) |
| RT only, n (%) | 31 | (14.9) |
| RP and RT, n (%) | 74 | (35.6) |
| At least 1 prior systemic | 58 | (27.9) |
| therapy, n (%) | | |
| Total Gleason Score, n (%) | | |
| <8 | 153 | (73.6) |
| ≥8 | 55 | (26.4) |
| PSA (ng/mL) (n = 202): | 0.8 | (0.17, 98.45) |
| Median (range) | | |
| PSA Group (n = 202), n (%) | | |
| <2.0 ng/mL | 139 | (68.8) |
| <0.5 ng/mL | 69 | (34.2) |
| 0.5 to <1.0 | 37 | (18.3) |
| ng/mL | | |
| 1.0 to <2.0 | 33 | (16.3) |
| ng/mL | | |
| ≥2.0 ng/mL | 63 | (31.2) |
| 2.0 to <5.0 | 33 | (16.3) |
| ng/mL | | |
| ≥5.0 ng/mL | 30 | (14.9) |
| 18F-DCFPyL dosing and uptake time | | |
| Administered activity (mCi): | 9.42 | (7.49-11.07) |
| median (range) | | |
| Time from injection to | 79 | (59-115) |
| imaging (minutes): median (range) | | |

Abbreviations:
PSA: Prostate-specific antigen;
RP: Radical prostatectomy;
RT: Radiation therapy

TABLE 7
Correct localization rate across three independent readers

| | All Patients (n = 208) | | | | | | | | |
|---|---|---|---|---|---|---|---|---|---|
| | Reader 1 | | 95% CI (%) | Reader 2 | | 95% CI (%) | Reader 3 | | 95% CI (%) |
| Negative PyL Scan | 71 | (34.1%) | (27.7, 40.6) | 84 | (40.4%) | (33.7, 47.1) | 85 | (40.9%) | (34.2, 47.5) |
| Positive PyL Scan | 137 | (65.9%) | (59.4, 72.3) | 124 | (59.6%) | (52.9, 66.3) | 123 | (59.1%) | (52.5, 65.8) |
| Unevaluable* | 33 | | | 24 | | | 24 | | |
| CLR (TP/TP + FP) | 89/104 | (85.6%) | (78.8, 92.3) | 87/100 | (87.0%) | (80.4, 93.6) | 84/99 | (84.8%) | (77.8, 91.9) |

*Some patients were unevaluable for the primary endpoint, either because no SOT was submitted (between 17-25 patients, depending on the central reader), or because the ¹⁸F-DCFPyL PET/CT scan was deemed a false negative based on lesion level co-localization (7-8 patients), which was not a component of the primary endpoint calculation (due in part to the lack of specificity of comparative conventional imaging modalities).

TABLE 8
Correct localization rate by standard of truth

| | | | | | | |
|---|---|---|---|---|---|---|
| SOT | Reader 1 | 95% CI (%) | Reader 2 | 95% CI (%) | Reader 3 | 95% CI (%) |

Histopathology (n = 31)

| Negative PyL Scan | 2 | (6.5%) | (0.8, 21.7) | 1 | (3.2%) | (0.0, 17.6) | 3 | (9.7%) | (2.6, 25.7) |
|---|---|---|---|---|---|---|---|---|---|
| Positive PyL Scan | 29 | (93.5%) | (84.9, 100) | 30 | (96.8%) | (90.6, 100) | 28 | (90.3%) | (79.9, 100) |
| Unevaluable* | 1 | | | 1 | | | 2 | | |
| CLR (TP/TP + FP) | 22/28 | (78.6%) | (63.4, 93.8) | 24/29 | (82.8%) | (69.0, 96.5) | 21/26 | (80.8%) | (65.6, 95.9) |

Correlative imaging (n = 100)

| Negative PyL Scan | 18 | (18.0%) | (10.5, 25.5) | 24 | (24.0%) | (15.6, 32.4) | 23 | (23.0%) | (14.8, 31.2) |
|---|---|---|---|---|---|---|---|---|---|
| Positive PyL Scan | 82 | (82.0%) | (74.5, 89.5) | 76 | (76.0%) | (67.6, 84.4) | 77 | (77.0%) | (68.8, 85.2) |
| Unevaluable* | 7 | | | 6 | | | 5 | | |
| CLR (TP/TP + FP) | 66/75 | (88.0%) | (80.6, 95.4) | 62/70 | (88.6%) | (81.1, 96.0) | 62/72 | (86.1%) | (78.1, 94.1) |

PSA response (n = 1)

| Negative PyL Scan | 0 | (0.0%) | N/A | 0 | (0.0%) | N/A | 0 | (0.0%) | N/A |
|---|---|---|---|---|---|---|---|---|---|
| Positive PyL Scan | 1 | (100%) | (16.7, 100) | 1 | (100%) | (16.7, 100) | 1 | (100%) | (16.7, 100) |
| Unevaluable* | 0 | | | 0 | | | 0 | | |
| CLR (TP/TP + FP) | 1/1 | (100%) | (16.7, 100) | 1/1 | (100%) | (16.7, 100) | 1/1 | (100%) | (16.7, 100) |

Abbreviations:
CLR: Correct localization rate;
PSA: Prostate-specific antigen;
SOT: Standard of truth.
*SOT not submitted or false negative at the lesion level

TABLE 9
Positive predictive value by standard of truth

| | | | | | | |
|---|---|---|---|---|---|---|
| SOT | Reader 1 | 95% CI (%) | Reader 2 | 95% CI (%) | Reader 3 | 95% CI (%) |

Histopathology (n = 31)

| | | | | | | | | | |
|---|---|---|---|---|---|---|---|---|---|
| Negative PyL Scan | 2 | (6.5%) | (0.8, 21.7) | 1 | (3.2%) | (0.0, 17.6) | 3 | (9.7%) | (2.6, 25.7) |
| Positive PyL Scan | 29 | (93.5%) | (84.9, 100) | 30 | (96.8%) | (90.6, 100) | 28 | (90.3%) | (79.9, 100) |
| PPV (TP/TP + FP) | 27/29 | (93.1%) | (83.9, 100) | 28/30 | (93.3%) | (84.4, 100) | 26/28 | (92.9%) | (83.3, 100) |

Correlative imaging (n = 100)

| | | | | | | | | | |
|---|---|---|---|---|---|---|---|---|---|
| Negative PyL Scan | 18 | (18.0%) | (10.5, 25.5) | 24 | (24.0%) | (15.6, 32.4) | 23 | (23.0%) | (14.8, 31.2) |
| Positive PyL Scan | 82 | (82.0%) | (74.5, 89.5) | 76 | (76.0%) | (67.6, 84.4) | 77 | (77.0%) | (68.8, 85.2) |
| PPV (TP/TP + FP) | 73/82 | (89.0%) | (82.3, 95.8) | 68/76 | (89.5%) | (82.6, 96.4) | 67/77 | (87.0%) | (79.5, 94.5) |

PSA response (n = 1)

| | | | | | | | | | |
|---|---|---|---|---|---|---|---|---|---|
| Negative PyL Scan | 0 | (0.0%) | N/A | 0 | (0.0%) | N/A | 0 | (0.0%) | N/A |
| Positive PyL Scan | 1 | (100%) | (16.7, 100) | 1 | (100%) | (16.7, 100) | 1 | (100%) | (16.7, 100) |
| PPV (TP/TP + FP) | 1/1 | (100%) | (16.7, 100) | 1/1 | (100%) | (16.7, 100) | 1/1 | (100%) | (16.7, 100) |

Abbreviations:
PPV: Positive predictive value;
PSA: Prostate-specific antigen;
SOT: Standard of truth

TABLE 10
Correct localization rate by correlative imaging standard of truth

| | | | | | | |
|---|---|---|---|---|---|---|
| Correlative imaging | | | | | | |
| SOT | Reader 1 | 95% CI (%) | Reader 2 | 95% CI (%) | Reader 3 | 95% CI (%) |

PET-Fluciclovine (n = 71)

| Negative PyL Scan | 10 | (14.1%) | (6.0, 22.2) | 17 | (23.9%) | (14.0, 33.9) | 14 | (19.7%) | (10.5, 29.0) |
|---|---|---|---|---|---|---|---|---|---|
| Positive PyL Scan | 61 | (85.9%) | (77.8, 94.0) | 54 | (76.1%) | (66.1, 86.0) | 57 | (80.3%) | (71.0, 89.5) |
| Unevaluable* | 6 | | | 4 | | | 4 | | |
| CLR (TP/TP + FP) | 50/55 | (90.9%) | (83.3, 98.5) | 45/50 | (90.0%) | (81.7, 98.3) | 46/53 | (86.8%) | (77.7, 95.9) |

MRI (n = 23)

| Negative PyL Scan | 7 | (30.4%) | (11.6, 49.2) | 7 | (30.4%) | (11.6, 49.2) | 7 | (30.4%) | (11.6, 49.2) |
|---|---|---|---|---|---|---|---|---|---|
| Positive PyL Scan | 16 | (69.6%) | (50.8, 88.4) | 16 | (69.6%) | (50.8, 88.4) | 16 | (69.6%) | (50.8, 88.4) |
| Unevaluable* | 1 | | | 1 | | | 1 | | |
| CLR (TP/TP + FP) | 12/15 | (80.0%) | (59.8, 100) | 13/15 | (86.7%) | (69.5, 100) | 13/15 | (86.7%) | (69.5, 100) |

CT (n = 6)

| Negative PyL Scan | 1 | (16.7%) | (1.1, 58.2) | 1 | (16.7%) | (1.1, 58.2) | 1 | (16.7%) | (1.1, 58.2) |
|---|---|---|---|---|---|---|---|---|---|
| Positive PyL Scan | 5 | (83.3%) | (53.5, 100) | 5 | (83.3%) | (53.5, 100) | 5 | (83.3%) | (53.5, 100) |
| Unevaluable* | 0 | | | 1 | | | 0 | | |
| CLR (TP/TP + FP) | 4/5 | (80.0%) | (36.0, 98.0) | 4/4 | (100.0%) | (45.4, 100) | 4/5 | (80.0%) | (36.0, 98.0) |

Abbreviations:
CLR: Correct localization rate;
PSA: Prostate-specific antigen;
SOT: Standard of truth
*SOT not submitted or false negative at the lesion level

TABLE 11
Baseline Conventional Imaging

| | All Patients, n (%) | |
|---|---|---|
| Imaging Type | (n = 208) | |
| ^{99m}Tc Whole-body Bone Scan | 131 | (63.0) |
| and CT/MRI/FDG PET | | |
| ^{99m}Tc whole-body bone scan | 119 | (57.2) |
| and CT | | |
| ^{99m}Tc whole-body bone scan | 10 | (4.8) |
| and MRI | | |
| ^{99m}Tc whole-body bone scan | 1 | (0.5) |
| and CT and MRI | | |
| ^{99m}Tc whole-body bone scan | 1 | (0.5) |
| and FDG PET | | |
| CT or MRI | 42 | (20.2) |
| ¹⁸F-fluciclovine or | 20 | (9.6) |
| ¹¹C-choline PET | | |
| ¹⁸F-fluciclovine PET alone | 16 | (7.7) |
| ¹⁸F-fluciclovine PET and MRI | 3 | (1.4) |
| ¹¹C-choline PET and MRI | 1 | (0.5) |
| ^{99m}Tc whole body bone scan only | 15 | (7.2) |

TABLE 12
CLR by baseline PSA levels

| | Reader 1 | | Reader 2 | | Reader 3 | |
|---|---|---|---|---|---|---|
| PSA (ng/mL) | n (%) | 95% CI | n (%) | 95% CI | n (%) | 95% CI |
| <0.5 | 11 (73.3) | (51.0, 95.7) | 10 (76.9) | (54.0, 99.8) | 11 (73.3) | (51.0, 95.7) |
| 0.5-<1.0 | 12 (75.0) | (53.8, 96.2) | 10 (76.9) | (54.0, 99.8) | 11 (73.3) | (51.0, 95.7) |
| 1.0-<2.0 | 17 (85.0) | (69.4, 100) | 14 (77.8) | (58.6, 97.0) | 15 (83.3) | (66.1, 100) |
| 2.0 < 5.0 | 21 (91.3) | (79.8, 100) | 24 (92.3) | (82.1, 100) | 20 (90.9) | (78.9, 100) |
| ≥5 | 27 (96.4) | (89.6, 100) | 28 (96.6) | (89.9, 100) | 26 (96.3) | (89.2, 100) |

TABLE 13
Detection rates by baseline PSA groups

| | Reader 1 | | Reader 2 | | Reader 3 | |
|---|---|---|---|---|---|---|
| PSA (ng/mL) | n (%) | 95% CI | n (%) | 95% CI | n (%) | 95% CI |
| <0.5 | 29 (42.0) | (30.4, 53.7) | 23 (33.3) | (22.2, 44.5) | 25 (36.2) | (24.9, 47.6) |
| 0.5-<1.0 | 21 (56.8) | (40.8, 72.7) | 17 (45.9) | (29.9, 62.0) | 19 (51.4) | (35.2, 67.5) |
| 1.0-<2.0 | 24 (72.7) | (57.5, 87.9) | 22 (66.7) | (50.6, 82.8) | 19 (57.6) | (40.7, 74.4) |
| 2.0 < 5.0 | 28 (84.8) | (72.6, 97.1) | 29 (87.9) | (76.7, 99.0) | 27 (81.8) | (68.7, 95.0) |
| ≥5 | 29 (96.7) | (90.2, 100) | 29 (96.7) | (90.2, 100) | 27 (90.0) | (79.3, 100) |

TABLE 14
PPV by anatomic region (prostatic, pelvic, and extra- pelvic))

| Anatomic | Reader 1 | | Reader 2 | | Reader 3 | |
|---|---|---|---|---|---|---|
| Region | n (%) | 95% CI | n (%) | 95% CI | n (%) | 95% CI |
| Prostatic | 31 (79.5) | (66.8, 92.2) | 30 (83.3) | (71.2, 95.5) | 27 (75.0) | (60.9, 89.1) |
| Pelvic | 41 (67.2) | (55.4, 79.0) | 40 (72.7) | (61.0, 84.5) | 39 (70.9) | (58.9, 82.9) |
| Extra-pelvic | 33 (67.3) | (54.2, 80.5) | 31 (67.4) | (53.8, 80.9) | 30 (69.8) | (56.0, 83.5) |

Abbreviations:
CI: Confidence interval;
PPV: Positive predictive value

TABLE 15
PPV by extra pelvic region

| Extra-pelvic | Reader 1 | | Reader 2 | | Reader 3 | |
|---|---|---|---|---|---|---|
| Region | n (%) | 95% CI | n (%) | 95% CI | n (%) | 95% CI |
| Lymph nodes (M1a) | 16 (61.5) | (42.8, 80.2) | 15 (65.2) | (45.8, 84.7) | 14 (60.9) | (40.9, 80.8) |
| Bone (M1b) | 15 (62.5) | (43.1, 81.9) | 14 (63.6) | (43.5, 83.7) | 14 (60.9) | (40.9, 80.8) |
| Visceral/soft | 2 (28.6) | (7.6, 64.8) | 2 (22.2) | (5.3, 55.7) | 2 (33.3) | (9.3, 70.4) |
| tissue (M1c) | | | | | | |

TABLE 16
Analysis of Change in Medical Management Questionnaire by Local Reader Response

| | | All Patients (n = 208) | | | | | |
|---|---|---|---|---|---|---|---|
| | | Local Reader | | | | | |
| | | Positive | 95% | Negative | 95% | | 95% |
| | | Scan | Confidence | Scan | Confidence | Total | Confidence |
| Parameter | Statistic | (N = 144) | Interval | (N = 63) | Interval | (N = 207) | Interval |

Any Change in Management

| | | | | | | | | | | |
|---|---|---|---|---|---|---|---|---|---|---|
| Yes | n (%) | 103 | (72.5%) | (64.4%, 79.7%) | 28 | (44.4%) | (31.9%, 57.5%) | 131 | (63.9%) | (56.9%, 70.5%) |
| No | n (%) | 39 | (27.5%) | (20.3%, 35.6%) | 35 | (55.6%) | (42.5%, 68.1%) | 74 | (36.1%) | (29.5%, 43.1%) |

| | | | | |
|---|---|---|---|---|
| Not Evaluable | n | 2 | 0 | 2 |

Change from No Treatment Planned to Any Treatment Planned

| | | | | | | | | | | |
|---|---|---|---|---|---|---|---|---|---|---|
| Yes | n (%) | 48 | (33.8%) | (26.1%, 42.2%) | 1 | (1.6%) | (0.0%, 8.5%) | 49 | (23.9%) | (18.2%, 30.3%) |
| No | n (%) | 94 | (66.2%) | (57.8%, 73.9%) | 62 | (98.4%) | (91.5%, 100%) | 156 | (76.1%) | (69.7%, 81.8%) |

| | | | | |
|---|---|---|---|---|
| Not Evaluable | n | 2 | 0 | 2 |

Change from Any Treatment Planned to No Treatment Planned

| | | | | | | | | | | |
|---|---|---|---|---|---|---|---|---|---|---|
| Yes | n (%) | 3 | (2.1%) | (0.4%, 6.0%) | 6 | (9.5%) | (3.6%, 19.6%) | 9 | (4.4%) | (2.0%, 8.2%) |
| No | n (%) | 139 | (97.9%) | (94.0%, 99.6%) | 57 | (90.5%) | (80.4%, 96.4%) | 196 | (95.6%) | (91.8%, 98.0%) |

| | | | | |
|---|---|---|---|---|
| Not Evaluable | n | 2 | 0 | 2 |

Change in Treatment Planned - Local Therapy to Systemic Therapy

| | | | | | | | | | | |
|---|---|---|---|---|---|---|---|---|---|---|
| Yes | n (%) | 40 | (28.2%) | (20.9%, 36.3%) | 18 | (28.6%) | (17.9%, 41.3%) | 58 | (28.3%) | (22.2%, 35.0%) |
| No | n (%) | 102 | (71.8%) | (63.7%, 79.1%) | 45 | (71.4%) | (58.7%, 82.1%) | 147 | (71.7%) | (65.0%, 77.8%) |

| | | | | |
|---|---|---|---|---|
| Not Evaluable | n | 2 | 0 | 2 |

Change in Treatment Planned - Systemic Therapy to Local Therapy

| | | | | | | | | | | |
|---|---|---|---|---|---|---|---|---|---|---|
| Yes | n (%) | 25 | (17.6%) | (11.7%, 24.9%) | 18 | (28.6%) | (17.9%, 41.3%) | 43 | (21.0%) | (15.6%, 27.2%) |
| No | n (%) | 117 | (82.4%) | (75.1%, 88.3%) | 45 | (71.4%) | (58.7%, 82.1%) | 162 | (79.0%) | (72.8%, 84.4%) |

| | | | | |
|---|---|---|---|---|
| Not Evaluable | n | 2 | 0 | 2 |

Change in Treatment Planned - Local Therapy to Other Therapy

| | | | | | | | | | | |
|---|---|---|---|---|---|---|---|---|---|---|
| Yes | n (%) | 0 | (0.0%) | (0.0%, 2.6%) | 0 | (0.0%) | (0.0%, 5.7%) | 0 | (0.0%) | (0.0%, 1.8%) |
| No | n (%) | 142 | (100%) | (97.4%, 100%) | 63 | (100%) | (94.3%, 100%) | 205 | (100%) | (98.2%, 100%) |

| | | | | |
|---|---|---|---|---|
| Not Evaluable | n | 2 | 0 | 2 |

95% CI = two-sided 95% confidence interval derived from a one-sample binomial distribution.
Responses may exist in subquestions under yes as several types of changes may exist.

### Example 10—Example Medical Management Questionnaires (MMQ)

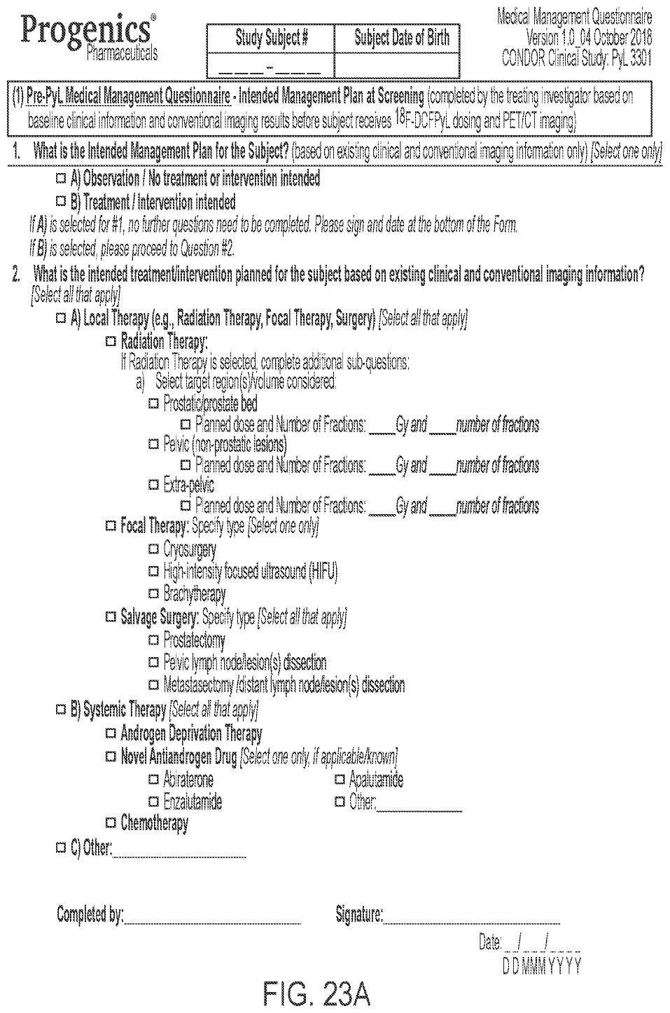

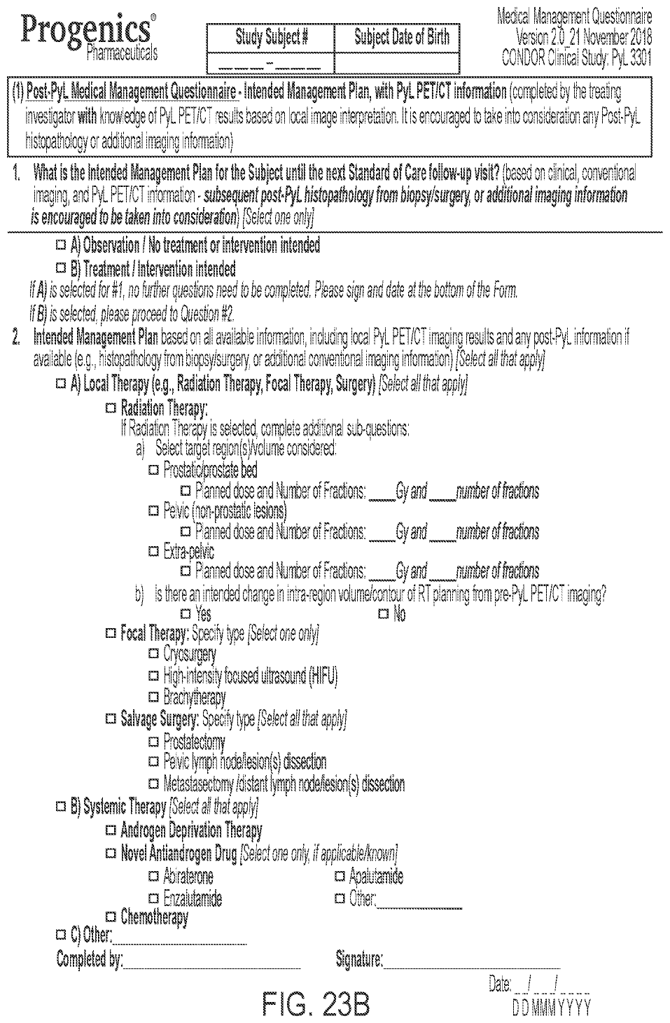

In any one of the methods provided herein, a pre- and/or post-MMQ questionnaire can be completed prior to and/or post imaging with ¹⁸F-DCFPyL PET/CT, respectively, as shown in FIGS. 23A-23B. Thus, any one of the methods provided herein can comprise a step of completing a pre- and/or post-MMQ questionnaire prior to and/or post imaging with ¹⁸F-DCFPyL PET/CT, respectively, as provided herein.

### Example 11—Performance in Patients with Recurrent or Metastatic Prostate Cancer

In CONDOR, 208 patients with elevated PSA after initial therapy and no radiologic evidence of cancer recurrence or metastasis based on conventional imaging underwent PyLARIFY imaging. Due to the absence of a lesion amenable to biopsy, central reader performance was compared against a composite truth standard of biopsy or surgery in 31 patients, correlative imaging in 100 patients and PSA response to therapy in one patient. The median baseline PSA was 0.8 ng/mL (inter-quartile range 0.39-3) with 67% of patients having a PSA <2.0 ng/mL prior to PyLARIFY imaging. PyLARIFY imaging detected at least one occult prostate cancer lesion in 59% (95% CI: 53, 66) to 66% (59, 72) of patients. The correct localization rate (95% CI) [i.e. PPV defined by anatomic lesion location matching] ranged from 85% (78, 92) to 87% (80, 94) on the patient level. PPV ranged from 77% to 78% in patients with baseline PSA <2 ng/mL and from 94% to 95% in PSA ≥2 ng/mL. Intended treatment management plans changed after PyLARIFY imaging in 64% of patients as evaluated by a questionnaire.

Inter-reader agreement for detection of recurrence or metastasis demonstrated a 76% concordance and Fleiss' kappa statistic (95% CI) of 0.65 (0.58, 0.73) across 208 patients.

In OSPREY, 117 patients with presumptive recurrent or metastatic prostate cancer based on conventional imaging underwent PyLARIFY imaging. Of these, 93 patients had an interpretable scan and pathology results from biopsy of the presumptive lesion. The median PSA was 7 ng/mL (range 0.03-597) with 32 (27%) patients with a PSA <2 ng/mL prior to PyLARIFY imaging.

The performance of the three central readers for detecting presumptive recurrent or metastatic prostate cancer ranged in sensitivity (95% CI) from 93% (84, 97) to 99% (92, 100) and PPV (95% CI) from 81% (73, 90) to 88% (80, 95). The detection of occult distant metastasis was 58% (19/33) of patients and 91% of these patients with an extrapelvic biopsy were confirmed by pathology.

In CONDOR and OSPREY, the PPV of PyLARIFY imaging for detecting prostate cancer recurrence or metastasis at the patient level and at the region levels are shown in Table 17.

TABLE 17
Positive Predictive Value (PPV) of PyLARIFY for Detecting Suspected Prostate
Cancer Recurrence or Metastasis, at the Patient Level and at the Prostatic,
Pelvic, and Extrapelvic Region Levels (OSPREY and CONDOR)

| | OSPREY Reader | | | CONDOR Reader | | |
|---|---|---|---|---|---|---|
| | 1 | 2 | 3 | 4 | 5 | 6 |
| Patient (N) | 92 | 93 | 92 | 137 | 124 | 123 |
| True Positive | 69 | 68 | 65 | 101 | 97 | 94 |
| False positive | 16 | 15 | 9 | 11 | 10 | 12 |
| PPV % (95% CI) | 81 | 82 | 88 | 90 | 91 | 89 |
| | (73, 90) | (74, 90) | (80, 95) | (85, 96) | (85, 96) | (83, 95) |
| Prostate bed | 0 | 0 | 0 | 39 | 36 | 36 |
| True Positive | 0 | 0 | 0 | 31 | 30 | 27 |
| False positive | 0 | 0 | 0 | 8 | 6 | 9 |
| PPV % (95% CI) | NE | NE | NE | 80 | 83 | 75 |
| | | | | (67,92) | (71, 96) | (61, 89) |
| Pelvic | 20 | 18 | 16 | 61 | 55 | 55 |
| True Positive | 15 | 14 | 15 | 41 | 40 | 39 |
| False positive | 5 | 4 | 1 | 20 | 15 | 16 |
| PPV % (95% CI) | 75 | 78 | 94 | 67 | 73 | 71 |
| | (53, 89) | (54, 92) | (70, 100) | (55, 79) | (61,85) | (59, 83) |
| Extrapelvic | 65 | 65 | 58 | 49 | 46 | 43 |
| True Positive | 54 | 54 | 50 | 33 | 31 | 30 |
| False positive | 11 | 11 | 8 | 16 | 15 | 13 |
| PPV % (95% CI) | 83 | 83 | 86 | 67 | 67 | 70 |
| | (74, 92) | (74, 92) | (77, 95) | (54, 81) | (54, 81) | (56, 84) |

N = number of positive patient scans evaluated with standard of truth;
PPV = positive predictive value;
NE = not evaluated

### Example 12—Performance in Patients with High-Risk Prostate Cancer

In OSPREY, 268 patients at high risk for pelvic lymph node metastasis who were planned for initial definitive therapy underwent PyLARIFY imaging. Of these, 252 patients had both an interpretable scan and surgical staging. PyLARIFY and baseline conventional imaging reader interpretations were compared to pathologic staging of pelvic nodal metastases. The results are presented in Table 18.

TABLE 18
Performance of PyLARIFY and Conventional Imaging* for Pelvic Lymph
Node Metastasis in Patients with High Risk Prostate Cancer (OSPREY)

| | Conventional | PyLARIFY Imaging | | |
|---|---|---|---|---|
| | Imaging* Reader | Reader 1 | Reader 2 | Reader 3 |
| Patient | N = 252 | N = 252 | N = 252 | N = 252 |
| True Positive | 26 | 26 | 19 | 25 |
| False Positive | 66 | 4 | 2 | 7 |
| True Negative | 123 | 186 | 188 | 183 |
| False Negative | 35 | 36 | 43 | 37 |
| Sensitivity % (95% CI) | 43 (30, 55) | 42 (30, 54) | 31 (19, 42) | 40 (28, 53) |
| PPV % (95% CI) | 28 (19, 38) | 87 (70, 95) | 91 (70, 99) | 78 (64, 93) |
| Specificity % (95% CI) | 65 (58, 72) | 98 (95, 99) | 99 (96, 100) | 96 (94, 99) |
| NPV % (95% CI) | 78 (71, 84) | 84 (80, 89) | 81 (76, 86) | 83 (78, 87) |

N = number of patients with pathology results from pelvic lymph node dissection; PPV = postivie predictive value; NPV = negative predictive value.
*Conventional imaging consisted of computer tomography, except for 3 cases of magnetic resonance imaging.

Inter-reader agreement for detection of pelvic lymph node metastasis was evaluated and demonstrated 92% concordance between readers with a generalized (Fleiss') kappa statistic of 0.78 (95% CI: 0.71, 0.85) across 268 patients.

### Example 13—a Prospective Phase 2/3 Study of PSMA-Targeted ¹⁸F-DCFPyL-PET/CT in Patients (Pts) with Prostate Cancer (PCa) (OSPREY): A Sub-Analysis of Disease Staging Changes in PCa Pts with Recurrence or Metastases on Conventional Imaging

### BACKGROUND

Conventional imaging and bone scintigraphy are suboptimal modalities for identifying PCa. PSMA-based imaging is highly promising for PCa detection. ¹⁸F-DCFPyL is a PSMA-targeted radiopharmaceutical for positron emission tomography (PET) that can be useful in staging of PCa. The diagnostic performance, detection rate, and potential impact of ¹⁸F-DCFPyL on staging of patients with high-risk PCa has been investigated. The impact of ¹⁸F-DCFPyL on staging of patients with PCa recurrence or metastases on conventional imaging has also been investigated.

Methods ¹⁸F-DCFPyL-PET/CT was evaluated in 117 men with radiologic evidence of local recurrence or metastatic disease on baseline anatomical imaging (CT, MRI) or whole-body bone scintigraphy and in whom at least one lesion was deemed amenable to biopsy. A single dose of 9 mCi (333 MBq) of ¹⁸F-DCFPyL was administered via intravenous injection, followed by PET/CT acquisition 1 to 2 hours thereafter. Based on TNM staging: prostatic (T), pelvic LN (N), extra-pelvic LN (M1a), bone (M1b) and other visceral organs/soft tissue (M1c), ¹⁸F-DCFPyL-PET/CT detection rates including lesion counts were systematically analyzed. Three central, blinded, and independent readers evaluated the ¹⁸F-DCFPyL scans.

### Results

In this study, 82 (70%) patients had baseline radiographic M1 stage disease (14 patients with M1a, 50 patients with M1b, 18 patients with M1c) and 33 (28%) patients were M0 stage at baseline by central conventional imaging review; two patients were unevaluable. ¹⁸F-DCFPyL-PET/CT up-staged 58% (19/33) of pts from M0 to M1, of whom 91% (10/11) who underwent an extra-pelvic biopsy were confirmed to have M1 disease by pathology, including 9 patients with M1b and 1 patient with M1a. Of the patients who were staged M1 at baseline, ¹⁸F-DCFPyL-PET/CT upstaged 16% (10/64; M1a to M1b or M1c: n=4; M1b to M1c: n=6) of pts to a higher M1 sub-stage and down-staged 22% (18/82) to M0.

### CONCLUSIONS

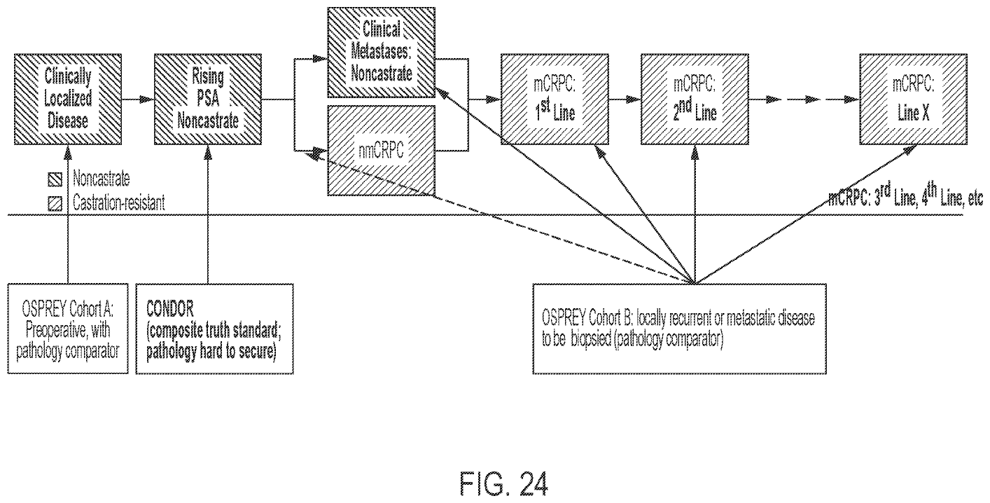

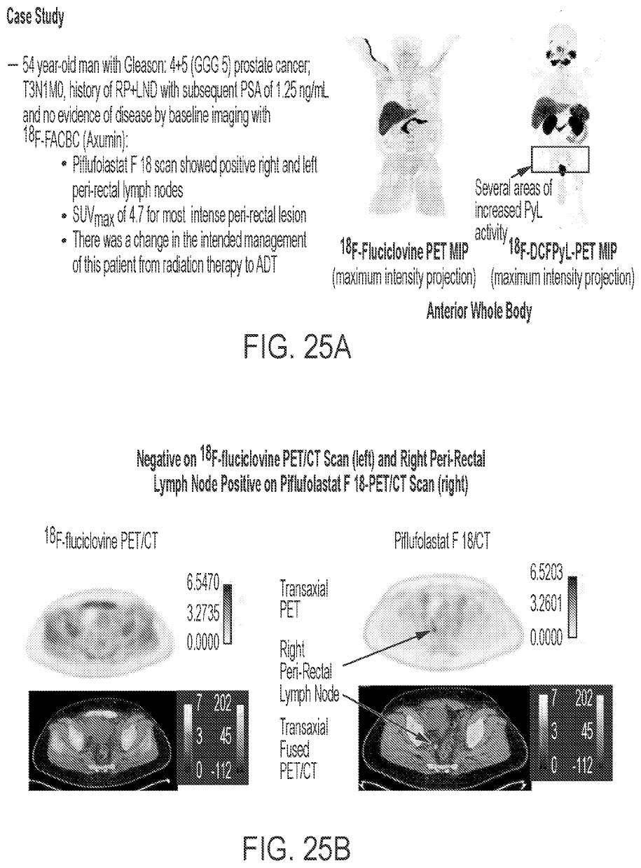

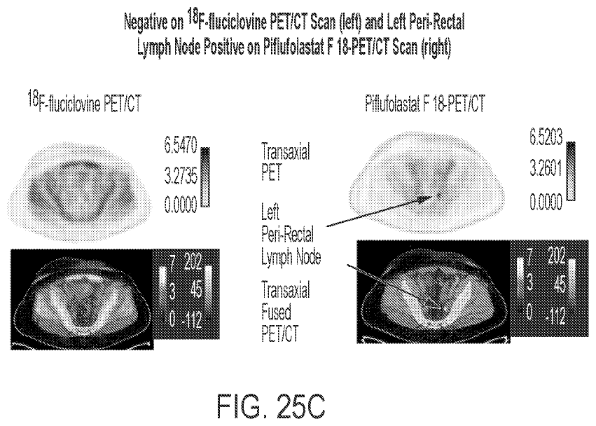

¹⁸F-DCFPyL-PET/CT identified M1 disease in the majority of patients examined who otherwise had locoregional disease. These data suggest that ¹⁸F-DCFPyL-PET/CT can be a useful tool in properly staging men with nonmetastatic relapsed disease, which could lead to superior treatment paradigms than using conventional imaging (FIG. 24).

### REFERENCES

1. Amling C L, Blute M L, Bergstralh E J, Seay T M, Slezak J, Zincke H. Long-term hazard of progression after radical prostatectomy for clinically localized prostate cancer: continued risk of biochemical failure after 5 years. J Urol 2000; 164: 101-105.
2. Han M, Partin A W, Zahurak M, Piantadosi S, Epstein J I, Walsh P C. Biochemical (prostate specific antigen) recurrence probability following radical prostatectomy for clinically localized prostate cancer. J Urol 2003; 169: 517-523.
3. Freedland S J, Humphreys E B, Mangold L A, et al. Risk of prostate cancer-specific mortality following biochemical recurrence after radical prostatectomy. JAMA 2005; 294: 433-439.
4. Kestin L L, Vicini F A, Ziaja E L, Stromberg J S, Frazier R C, Martinez A A. Defining biochemical cure for prostate carcinoma patients treated with external beam radiation therapy. Cancer 1999; 86: 1557-1566.
5. Paller C J, Antonarakis E S. Management of biochemically recurrent prostate cancer after local therapy: evolving standards of care and new directions. Clin Adv Hematol Oncol 2013; 11: 14-23.
6. Song H, Harrison C, Duan H, et al. Prospective evaluation in an academic center of ¹⁸F-DCFPyL PET/CT in biochemically recurrent prostate cancer: a focus on localizing disease and changes in management. J Nucl Med 2019; published online October 18. DOI:10.2967/jnumed.119.231654.
7. Shinohara K, Wheeler T M, Scardino P T. The appearance of prostate cancer on transrectal ultrasonography: correlation of imaging and pathological examinations. J Urol 1989; 142: 76-82.
8. Scheidler J, Hricak H, Vigneron D B, et al. Prostate cancer: localization with three-dimensional proton M R spectroscopic imaging—clinicopathologic study. Radiology 1999; 213: 473-480.
9. Blomqvist L, Carlsson S, Gjertsson P, et al. Limited evidence for the use of imaging to detect prostate cancer: a systematic review. Eur J Radiol 2014; 83: 1601-1606.
10. Schuster D M, Nieh P T, Jani A B, et al. Anti-3[¹⁸F]FACBC positron emission tomography-computerized tomography and ¹¹¹In-capromab pendetide single photon emission computerized tomography-computerized tomography for recurrent prostate carcinoma: results of a prospective clinical trial. J Urol 2014; 191: 1446-53.
11. Calais J, Ceci F, Eiber M, et al. ¹⁸F-fluciclovine PET-CT and ⁶⁸Ga-PSMA-11 PET-CT in patients with early biochemical recurrence after prostatectomy: a prospective, single centre, single-arm, comparative imaging trial. Lancet Oncol 2019; 20: 1286-1294.
12. Nanni C, Zanoni L, Pultrone C, et al. ¹⁸F-FACBC (anti1-amino-3-¹⁸F-fluorocyclobutane-1-carboxylic acid) versus ¹¹C-choline PET/CT in prostate cancer relapse: results of a prospective trial. Eur J Nucl Med Mol Imaging 2016; 43:1601-1610.
13. Dietlein M, Kobe C, Kuhnert G, et al. Comparison of [¹⁸F]DCFPyL and [⁶⁸Ga]Ga-PSMA-HBED-CC for PSMA-PET imaging in patients with relapsed prostate cancer. Mol Imaging Biol 2015; 17: 575-584.
14. Rowe S P, Macura K J, Mena E, et al. PSMA-based [¹⁸F]DCFPyL PET/CT is superior to conventional imaging for lesion detection in patients with metastatic prostate cancer. Mol Imaging Biol 2016; 18: 411-419.
15. Tan N, Oyoyo U, Bavadian N, et al. PSMA-targeted radiotracers versus ¹⁸F fluciclovine for the detection of prostate cancer biochemical recurrence after definitive therapy: a systematic review and meta-analysis. Radiology 2020; 296: 44-55.
16. Albisinni S, Artigas C, Aoun F, et al. Clinical impact of ⁶⁸Ga-prostate-specific membrane antigen (PSMA) positron emission tomography/computed tomography (PET/CT) in patients with prostate cancer with rising prostate-specific antigen after treatment with curative intent: preliminary analysis of a multidisciplinary approach. BJU Int 2017; 120: 197-203.
17. Hope T A, Aggarwal R, Chee B, et al Impact of ⁶⁸Ga-PSMA-11 PET on management in patients with biochemically recurrent prostate cancer. J Nucl Med 2017; 58: 1956-1961.
18. Chen Y, Pullambhatla M, Foss C A, et al. 2-(3-{1-Carboxy-5-[(6-[¹⁸F] fluoro-pyridine-3-carbonyl)-amino]-pentyl}-ureido)-pentanedioic acid, [¹⁸F] DCFPyL, a PSMA-based PET imaging agent for prostate cancer. Clin Cancer Res 2011 17: 7645-7653.
19. Szabo Z, Mena E, Rowe S P, et al. Initial evaluation of [¹⁸F]DCFPyL for prostate-specific membrane antigen (PSMA)-targeted PET imaging of prostate cancer. Mol Imaging Biol 2015; 17:565-574.
20. Gorin M A, Rowe S P, Patel H D, et al. Prostate specific membrane antigen targeted ¹⁸F-DCFPyL positron emission tomography/computerized tomography for the preoperative staging of high risk prostate cancer: results of a prospective, phase II, single center study. J Urol 2018; 199: 126-132.
21. Pienta K J, Gorin M A, Rowe S P, et al. A phase 2/3 prospective multicentre study of the diagnostic accuracy of prostate-specific membrane antigen (PSMA) PET/CT with ¹⁸F-DCFPyL in patients with prostate cancer (OSPREY). Submitted
22. Cookson, M S, Aus G, Burnett A L, et al. Variation in the definition of biochemical recurrence in patients treated with localized prostate cancer: the American Urological Association (AUA) prostate guidelines for localized prostate cancer update panel report and recommendation for a standard in the reporting of surgical outcomes. J Urol 2007; 177: 540-545.
23. Roach M 3rd, Hanks G, Thames H Jr, et al. Defining biochemical failure following radiotherapy with or without hormonal therapy in men with clinically localized prostate cancer: recommendations of the RTOG-ASTRO Phoenix Consensus Conference. Int J Radiat Oncol Biol Phys 2006; 65: 965-974.
24. Emmett L, Metser U, Bauman G, et al. Prospective, multisite, international comparison of ¹⁸F-fluoromethylcholine PET/CT, multiparametric MRI, and ⁶⁸Ga-HBED-CC PSMA-11 PET/CT in men with high-risk features and biochemical failure after radical prostatectomy: clinical performance and patient outcomes. J Nucl Med 2019; 60: 794-800.
25. Rowe S P, Campbell S P, Mana-Ay M, et al. Prospective evaluation of PSMA-targeted ¹⁸F-DCFPyL PET/CT in men with biochemical failure after radical prostatectomy for prostate cancer. J Nucl Med 2020; 61: 58-61.
26. Mena E, Lindenberg M L, Turkbey I B, et al. ¹⁸F-DCFPyL PET/CT imaging in patients with biochemical recurrence prostate cancer after primary local therapy. J Nucl Med 2020; 61: 881-889.
27. Wright G L, Haley C, Beckett M L, Schellhammer P F. Expression of prostate-specific membrane antigen in normal, benign, and malignant prostate tissues. Urol Oncol 1995; 1: 18-28
28. Ferreira G, Iravani A, Hofman M S, Hicks R J. Intra-individual comparison of ⁶⁸Ga-PSMA-11 and ¹⁸F-DCFPyL normal-organ biodistribution. Cancer Imaging 2019; 19: 23.
29. Werner R A, Derlin T, Lapa C, et al. ¹⁸F-labeled, PSMA-targeted radiotracers: leveraging the advantages of radiofluorination for prostate cancer molecular imaging Theranostics 2020; 10: 1-16.
30. Sanchez-Crespo A. Comparison of gallium-68 and fluorine-18 imaging characteristics in positron emission tomography. Appl Radiat Isot 2013; 76: 55-62.
31. Buyyounouski, M K et al. Prostate Cancer—Major changes in the American Joint Committee on Cancer Eight Edition Cancer Staging Manual. CA Cancer J Clin. 2017; 67(3):245-253.

1. A method of treating, or making a treatment management decision for, prostate cancer in a subject, the method comprising:
imaging the subject with ¹⁸F-DCFPyL PET/CT, and
treating or making a treatment management decision for the subject based on the imaging,
wherein the subject is one with metastatic or recurrent prostate cancer, or is suspected of having metastatic or recurrent prostate cancer; or has biochemically recurrent prostate cancer, or is suspected of having biochemically recurrent prostate cancer; or has a PSA level of less than 2 ng/ml; or has nonmetastatic relapsed prostate cancer, or is suspected of having nonmetastatic relapsed prostate cancer.

2. The method of claim 1, wherein the imaging is for determining the presence or absence of one or more prostate cancer lesions, such as one or more metastases, and the treating or making a treatment management decision is based on the presence or absence of the one or more prostate cancer lesions, such as one or more metastases.

3. The method of claim 1, wherein the imaging is for determining the stage of the prostate cancer in the subject.

4. The method of claim 1, wherein the subject has stage T, N (e.g., N1) or M1 (e.g., M1a, M1b, M1c) prostate cancer.

5-11. (canceled)

12. The method of claim 1, wherein ¹⁸F-DCFPyL is administered 1-2 hours prior to PET/CT to the subject.

13. (canceled)

14. The method of claim 1, wherein the method further comprises treating the subject based on the determining, optionally wherein the treatment is any one of the treatments provided herein.

15-29. (canceled)

30. The method of claim 1, wherein the method further comprises a step of performing an additional diagnostic test on the subject, such as a PSA test or conventional imaging test.

31-34. (canceled)

35. The method of claim 1, wherein the method further comprises completing pre- and/or post-imaging a questionnaire, such as of Example 10, respectively.

36. The method of claim 1, wherein the diagnostic performance of the ¹⁸F-DCFPyL PET/CT imaging, such as when centrally read, compared to centrally read conventional imaging (e.g., for determining the presence or absence of one or more prostate cancer lesions, such as one or more metastases, or determining the stage of the prostate cancer in the subject) has similar or increased PPV, NPV, specificity, and/or sensitivity.

37-38. (canceled)

39. The method of claim 1, wherein the diagnostic performance of the ¹⁸F-DCFPyL PET/CT imaging, such as when centrally read, compared to centrally read conventional imaging for determining pelvic lymph node metastases (N1) demonstrates at least 2-fold or 3-fold higher PPV, improved NPV, high or near-perfect specificity (e.g., at least 95%, 96% or 97%, or 97.9% vs. 67.3%, respectively), and/or similar or greater sensitivity (e.g., ˜40% or at least 40%).

40. (canceled)

41. A method of using ¹⁸F-DCFPyL to image men with prostate cancer, comprising:
(i) contacting a subject, such as any one of the subjects provided herein, with ¹⁸F-DCFPyL or PyLARIFY, and
(ii) imaging the subject, such as with PET/CT imaging;
wherein a study of diagnostic performance of the ¹⁸F-DCFPyL PET/CT imaging, such as when centrally read, compared to centrally read conventional imaging (e.g., for determining the presence or absence of one or more prostate cancer lesions, such as one or more metastases, or determining the stage of the prostate cancer in the subject) has similar or increased PPV, NPV, specificity, and/or sensitivity.

42-45. (canceled)

46. A method of restaging prostate cancer in a subject, said prostate cancer initially staged with conventional imaging, the method comprising:
imaging the subject with ¹⁸F-DCFPyL PET/CT, and
restaging the prostate cancer in the subject based on the imaging with ¹⁸F-DCFPyL PET/CT,
wherein the subject is one with metastatic or recurrent prostate cancer, or is suspected of having metastatic or recurrent prostate cancer; or has biochemically recurrent prostate cancer, or is suspected of having biochemically recurrent prostate cancer; or has nonmetastatic relapsed prostate cancer, or is suspected of having nonmetastatic relapsed prostate cancer.

47-49. (canceled)

50. The method of claim 46, wherein the method further comprises treating or making a treatment management decision for the subject based on the restage determined by the imaging with ¹⁸F-DCFPyL PET/CT and/or the comparing.

51-63. (canceled)

64. The method of claim 46, wherein the method further comprises treating the subject based on the restage determined by the imaging with ¹⁸F-DCFPyL PET/CT and/or the comparing.

65-67. (canceled)

68. The method of claim 46, wherein the method further comprises completing pre- and/or post-imaging a questionnaire, such as of Example 10, respectively.

69. ¹⁸F-DCFPyL for use in a method of treating, or making a treatment management decision for, prostate cancer in a subject, the method comprising:
imaging the subject with ¹⁸F-DCFPyL PET/CT, and treating or making a treatment management decision for the subject based on the imaging,
wherein the subject is one with metastatic or recurrent prostate cancer, or is suspected of having metastatic or recurrent prostate cancer; or has biochemically recurrent prostate cancer, or is suspected of having biochemically recurrent prostate cancer; or has a PSA level of less than 2 ng/ml; or has nonmetastatic relapsed prostate cancer, or is suspected of having nonmetastatic relapsed prostate cancer.

70. (canceled)

71. ¹⁸F-DCFPyL for use in a method of imaging men with prostate cancer, comprising: (i) contacting a subject, such as any one of the subjects provided herein, with ¹⁸F-DCFPyL, and (ii) imaging the subject, such as with PET/CT imaging.

72-73. (canceled)

74. ¹⁸F-DCFPyL for use in a method of restaging prostate cancer in a subject, said prostate cancer initially staged with conventional imaging, the method comprising:
imaging the subject with ¹⁸F-DCFPyL PET/CT, and
restaging the prostate cancer in the subject based on the imaging with ¹⁸F-DCFPyL PET/CT,
wherein the subject is one with metastatic or recurrent prostate cancer, or is suspected of having metastatic or recurrent prostate cancer; or has biochemically recurrent prostate cancer, or is suspected of having biochemically recurrent prostate cancer; or has nonmetastatic relapsed prostate cancer, or is suspected of having nonmetastatic relapsed prostate cancer.

75. (canceled)

76. ¹⁸F-DCFPyL for use according to claim 1, wherein the ¹⁸F-DCFPyL is in the form of a sterile, clear, colourless solution for intravenous injection having a pH of 4.5 to 7.5, each milliliter of which contains: <1 microgram of DCFPyL, 37 to 2960 MBq (1 to 80 mCi) florcarpiroic F 18 at calibration time and date, and ≤78.9 mg ethanol in 0.9% sodium chloride injection USP.

77. Use of ¹⁸F-DCFPyL for the manufacture of a medicament for use in a method as defined in claim 1.
